(19) United States
(12) Patent Application Publication
(10) Pub. No.: US 2022/0152397 A1
(43) Pub. Date: May 19, 2022

(54) NEUROSTIMULATION EVALUATION, PROGRAMMING AND CONTROL BASED ON SENSED BLOOD FLOW

(71) Applicant: Medtronic, Inc., Minneapolis, MN (US)

(72) Inventors: Brooke G. Kelley, Brooklyn Center, MN (US); Sean M. White, Rancho Santa Margarita, CA (US); Brian A. Smith, Apple Valley, MN (US); Anders Johan Magnus Johansson, Oak Park, CA (US); Melissa Y. Campos, Santa Monica, CA (US); Cassandra Elizabeth Morris, Plymouth, MN (US)

(21) Appl. No.: 17/454,760

(22) Filed: Nov. 12, 2021

Related U.S. Application Data
(60) Provisional application No. 63/114,364, filed on Nov. 16, 2020.
(60) Provisional application No. 63/114,358, filed on Nov. 16, 2020.
(60) Provisional application No. 63/136,347, filed on Jan. 12, 2021.
(60) Provisional application No. 63/136,343, filed on Jan. 12, 2021.

(51) Int. Cl. A61N 1/36; A61N 1/02; G16H 20/30

(52) U.S. Cl. CPC A61N 1/36139; A61N 1/36062; A61N 1/025; A61N 1/36185; G16H 20/30; A61N 1/36153; A61N 1/36157; A61N 1/36071

(57) ABSTRACT

A neurostimulation device, external programmer, or remote programming device may receive blood flow information relating to blood flow values from one or more blood flow sensing devices, either directly or via network connections, and perform, direct or control, based on the blood flow information, generation of neurostimulation efficacy information, information to assist in programming of one or more neurostimulation parameter, and/or automatic control of one or more neurostimulation stimulation parameters.

This application claims the benefit of U.S. Provisional Patent Application No. 63/114,358, filed on Nov. 16, 2020, U.S. Provisional Patent Application No. 63/114,364, filed on Nov. 16, 2020, U.S. Provisional Patent Application No. 63/136,343, filed on Jan. 12, 2021, and U.S. Provisional Patent Application No. 63/136,347, filed on Jan. 12, 2021, the entire contents of each of which are incorporated herein by reference.

### TECHNICAL FIELD

This disclosure generally relates to medical devices, and more specifically, electrical stimulation.

### BACKGROUND

Electrical stimulation devices, sometimes referred to as neurostimulators or neurostimulation devices, may be external to or implanted within a patient, and configured to deliver electrical stimulation therapy to various tissue sites to treat a variety of symptoms or conditions such as chronic pain, tremor, Parkinson's disease, epilepsy, or other neurological disorders, urinary or fecal incontinence, sexual dysfunction, obesity, or gastroparesis. An electrical stimulation device may deliver electrical stimulation therapy via electrodes, e.g., carried by one or more leads, positioned proximate to target locations associated with the brain, the spinal cord, pelvic nerves, tibial nerves, peripheral nerves, the gastrointestinal tract, or elsewhere within a patient. Stimulation proximate the spinal cord, proximate the sacral nerve, within the brain, and proximate peripheral nerves is often referred to as spinal cord stimulation (SCS), sacral neuromodulation (SNM), deep brain stimulation (DBS), and peripheral nerve stimulation (PNS), respectively.

A physician or clinician may select values for a number of programmable stimulation parameters in order to define the electrical stimulation therapy to be delivered by the implantable stimulator to a patient. For example, the physician or clinician may select one or more electrodes, polarities of selected electrodes, a voltage or current amplitude, a pulse width, and a pulse frequency as stimulation parameters. A set of therapy stimulation parameters, such as a set including electrode combination, electrode polarity, amplitude, pulse width and pulse frequency, may be referred to as a therapy program in the sense that they define the electrical stimulation therapy to be delivered to the patient.

### SUMMARY

In general, the disclosure describes techniques for neurostimulation efficacy evaluation, programming and/or control based on information relating to sensed blood flow. In some examples, sensed blood flow information may be used to evaluate efficacy of neurostimulation or neurostimulation stimulation parameters, such as electrode positions, electrode combinations, electrode polarities, stimulation amplitude, stimulation pulse width, stimulation pulse rate, and/or stimulation cycling, assist a user in evaluating efficacy of one or more of such stimulation parameters, assist a user in programming one or more of such stimulation parameters, and/or automatically control one or more of such stimulation parameters, e.g., on a closed loop basis. The sensed blood flow may be used as a biomarker to indicate correct lead placement and efficacy of stimulation parameters.

A blood flow sensing device may be positioned to sense blood flow in a tissue region as an indication of efficacy of neurostimulation in alleviating a disease, disorder or syndrome. As an example, levels of blood flow may be correlated with symptoms of painful diabetic neuropathy (PDN), peripheral vascular disease (PVD), peripheral arterial disease (PAD), complex regional pain syndrome (CRPS), angina pectoris (AP), or other diseases, disorders or syndromes, and thereby be indicative of efficacy of neurostimulation in eliminating or alleviating such symptoms, e.g., in a patient's toes or feet, or elsewhere in a patient's body. In some examples, neurostimulation with stimulation parameters selected, adjusted and/or controlled based on sensed blood flow information may be effective in preventing or delaying onset of aspects of a disease, disorder, or syndrome, such as, e.g., nerve damage or degeneration.

A neurostimulation device, external programmer, or remote programming device may receive blood flow information relating to blood flow values from one or more blood flow sensing devices, either directly or via network connections, and perform, direct or control, based on the blood flow information, generation of neurostimulation efficacy information, information to assist in programming of one or more neurostimulation stimulation parameters, and/or automatic control of one or more neurostimulation stimulation parameters. In this manner, a neurostimulation device, external programmer, or remote programming device may select, adjust or control one or more neurostimulation stimulation parameters based on the sensed blood flow information to eliminate or alleviate, or delay the onset of, symptoms of a disease, disorder or syndrome, or delay onset of tissue damage or degeneration.

In one example, a system comprises one or more processors configured to direct delivery of electrical stimulation to a patient, receive information relating to blood flow associated with tissue of the patient upon the delivery of the electrical stimulation to the patient, and generate output based on the received information.

In another example, a method comprises directing delivery of electrical stimulation with one or more processors to a patient, receiving information relating to blood flow associated with tissue of the patient upon the delivery of the electrical stimulation to the patient, and generating output based on the received information.

In another, a computer-readable medium comprises instructions to cause one or more processors to direct delivery of electrical stimulation to a patient, receive information relating to blood flow associated with tissue of the patient upon the delivery of the electrical stimulation to the patient, and generate output based on the received information.

In one example, a system comprises electrical stimulation circuitry configured to generate electrical stimulation, electrodes configured to deliver the electrical stimulation to a patient, and processing circuitry configured to receive information relating to blood flow associated with tissue of the patient, control the electrical stimulation circuitry to deliver the electrical stimulation to the patient based on the received information.

In another example, a method comprises generating electrical stimulation with electrical stimulation circuitry, delivering the electrical stimulation with electrodes to a patient, receiving information relating to blood flow associated with tissue of the patient upon delivering the electrical stimulation to the patient, and controlling the electrical stimulation circuitry to deliver the electrical stimulation to the patient based on the received information.

In another, a computer-readable medium comprises instructions to cause one or more processors to receive information relating to blood flow associated with tissue of the patient, and to control the electrical stimulation circuitry to deliver the electrical stimulation to the patient based on the received information

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes an implantable medical device (IMD) in the form of a neurostimulation device configured to deliver spinal cord stimulation (SCS), an external programmer, and one or more blood flow sensing devices in accordance with one or more techniques of this disclosure.

FIG. 2A is a block diagram illustrating an example of an 1 MB in the form of a neurostimulation device, in accordance with one or more techniques of this disclosure.

FIG. 2B is a block diagram illustrating an example of an 1 MB in the form of a neurostimulation device, in accordance with one or more techniques of this disclosure.

FIG. 3 is a block diagram illustrating an example of an external programmer suitable for use with the IMD of FIG. 2, in accordance with one or more techniques of this disclosure.

FIG. 4 is a block diagram illustrating an example of a blood flow sensing system suitable for use with the IMD of FIG. 2 and the programmer of FIG. 3, in accordance with one or more techniques of this disclosure.

FIG. 5 is a conceptual diagram of the blood flow sensing device of FIG. 4.

FIG. 6 is a block diagram of a system for evaluating efficacy of, assisting a user in programming, and/or automatically controlling neurostimulation or neurostimulation stimulation parameters.

FIG. 7 is a user interface diagram of an external programmer or remote monitoring/programming device suitable for use in the system of FIG. 6.

FIG. 8A is a flow diagram illustrating delivering neurostimulation while receiving sensed blood flow information.

FIG. 8B is a flow diagram illustrating controlling neurostimulation based on sensed blood flow information.

FIG. 8C is a flow diagram illustrating determining efficacy of neurostimulation based on sensed blood flow information.

FIG. 9 is a flow diagram illustrating programming of one or more neurostimulation stimulation parameters based on sensed blood flow information.

FIG. 10 is a flow diagram illustrating automated review of one or more neurostimulation stimulation parameters versus sensed blood flow information to support programming of neurostimulation stimulation parameters.

FIG. 11 is a flow diagram illustrating automated control of one or more neurostimulation stimulation parameters based on sensed blood flow information.

FIG. 12 is a flow diagram illustrating generation of index information based on correlation of one or more neurostimulation stimulation parameters and sensed blood flow information.

### DETAILED DESCRIPTION

Efficacy of neurostimulation in eliminating or alleviating symptoms, or preventing or delaying onset or progression of aspects of, a disease, disorder or syndrome, may vary according to the stimulation parameters used to deliver the neurostimulation to a patient. Selection of electrode positions relative to a neural target, as one example, can elicit a desired response to the neurostimulation. In the case of pain relief, the desired response to some types of neurostimulation can be a coincidence of paresthesia sensation with an area of pain.

Pain from conditions having an underlying autonomic or vascular dysfunction may coincide with blood flow changes in tissue in a target anatomical area. A blood flow sensing device can be used to detect changes in blood flow upon delivering neurostimulation with different neurostimulation stimulation parameters, such as different electrode positions, combinations and/or polarities, or different stimulation amplitudes, pulse widths, pulse rates, duty cycle, or patient data, such as posture, glucose level, time of day, or pain level indication. In some examples, sensed blood flow information may be used to support evaluation of neurostimulation efficacy, programming of neurostimulation programmers, delivery of stimulation, and/or automated control of neurostimulation stimulation parameters.

This disclosure describes techniques for neurostimulation efficacy evaluation, programming and/or control based on information relating to sensed blood flow. In some examples, sensed blood flow information may be used to evaluate efficacy of neurostimulation or neurostimulation stimulation parameters, such as lead position, electrode positions, combinations and polarities, or stimulation amplitude, pulse width, pulse rate, or cycling, assist a user in programming one or more of such stimulation parameters, and/or automatically control one or more of such stimulation parameters, e.g., on a closed loop basis. Using the sensed blood flow information to confirm electrode placement and/or device programming provides objective feedback to the clinician without requiring feedback from the patient, which can be particularly useful during surgery, or when a patient is asleep. In addition, the feedback from the sensed blood flow information allows for objective feedback for the clinician without having to engage or prompt the patient.

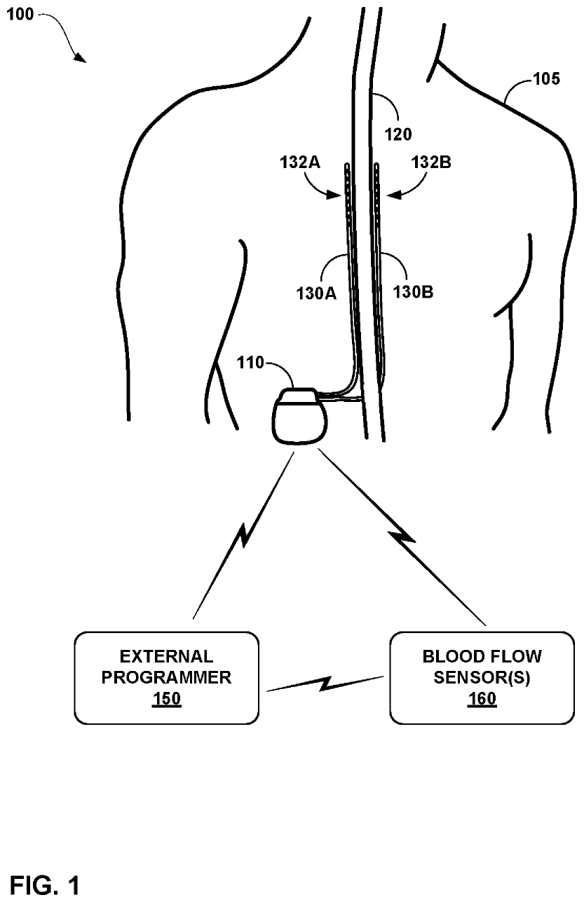

Systems and methods for neurostimulation efficacy evaluation, programming and/or control based on information relating to sensed blood flow are described herein. The system may include a stimulator system that interacts with a stimulator programmer, along with a blood flow detecting device. FIG. 1 is a conceptual diagram illustrating an example system 100 that includes an implantable medical device (IMD) 110 configured to deliver spinal cord stimulation (SCS) therapy, processing circuitry 140, an external programmer 150, and one or more blood flow sensors 160, in accordance with one or more examples of this disclosure. Processing circuitry 140 may include one or more processors configured to perform various operations of IMD 110. Although the examples described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of neurostimulation devices or other therapeutic applications of neurostimulation, including an external neurostimulator. For example, the system may not be a fully implanted system where the pulse generator is external to the patient and stimulation is transmitted transdermally. In one or more examples, the stimulators may be configured to deliver peripheral nerve stimulation or spinal nerve root stimulation.

As shown in FIG. 1, system 100 includes an IMD 110, leads 130A and 130B, and external programmer 150 shown in conjunction with a patient 105, who is ordinarily a human patient. In the example of FIG. 1, IMD 110 is an implantable electrical stimulator that is configured to generate and deliver electrical stimulation therapy to patient 105, e.g., for relief of chronic pain or other symptoms, via one or more electrodes 132A, 132B of leads 130A and/or 130B, respectively. In the example of FIG. 1, each lead 130A, 130B includes eight electrodes 132A, 132B respectively, although the leads may each have a different number of electrodes. Leads 130A, 130B may be referred to collectively as “leads 130” and electrodes 132A, 132B may be referred to collectively as electrodes 132. In other examples, IMD 110 may be coupled to a single lead carrying multiple electrodes or more than two leads each carrying multiple electrodes.

IMD 110 may be a chronic electrical stimulator that remains implanted within patient 105 for weeks, months, or years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to one or more leads percutaneously implanted within the patient. In some examples, IMD 110 uses electrodes on one or more leads, while in other examples, IMD 110 may use one or more electrodes on a lead or leads and one of more electrodes on a housing of the IMD. In further examples, IMD 110 may be leadless and instead use only electrodes carried on a housing of the IMD.

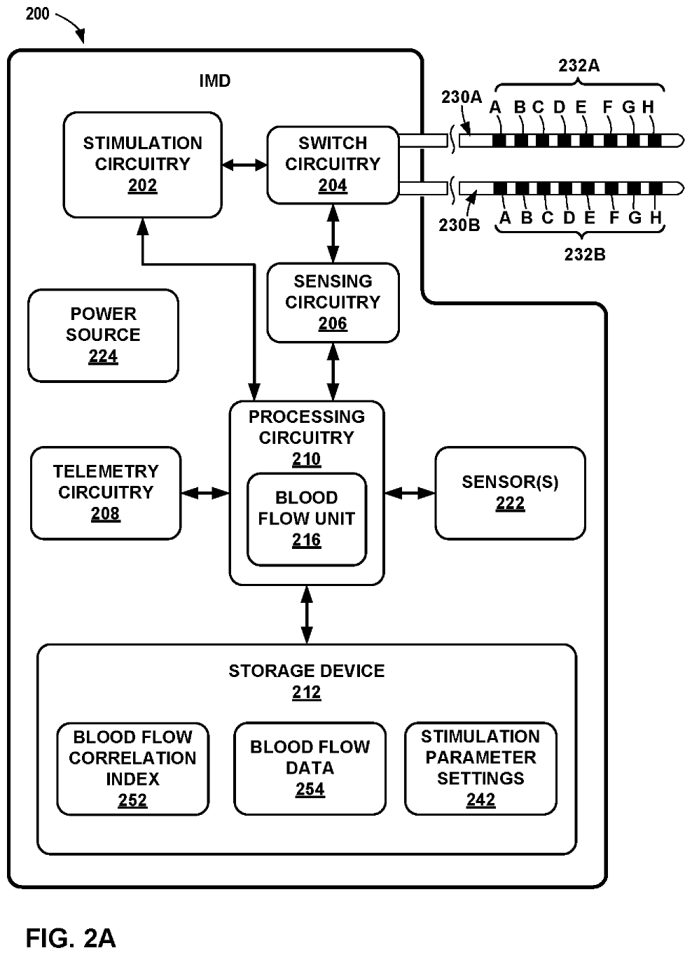

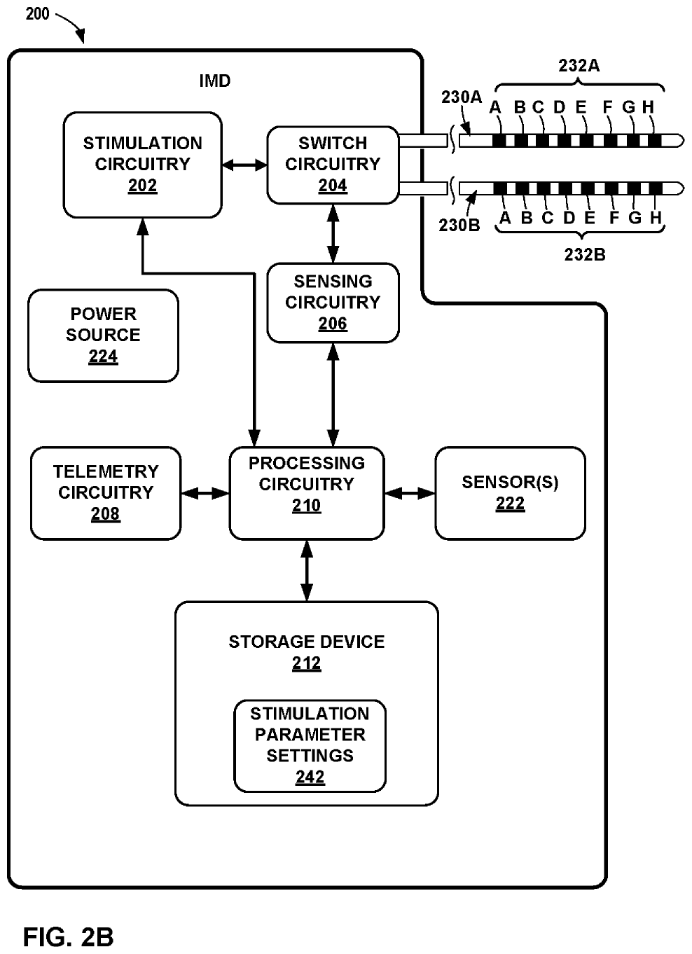

IMD 110 may be constructed of any polymer, metal, or composite material sufficient to house the components of IMD 110 (e.g., components illustrated in FIG. 2A, 2B) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted at other suitable sites within patient 105, which may depend, for example, on the target site within patient 105 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source.

In the example of FIG. 1, electrical stimulation energy, which may be delivered as regulated current or regulated voltage-based pulses, is delivered from IMD 110 to one or more target tissue sites of patient 105 via leads 130 and electrodes 132. Leads 130 position electrodes 132 adjacent to target tissue of spinal cord 120. One or more of the electrodes 132 may be disposed at a distal tip of a lead 130 and/or at other positions at intermediate points along the lead. Leads 130 may be implanted and coupled to IMD 110. The electrodes 132 may transfer electrical stimulation generated by an electrical stimulation generator in IMD 110 to tissue of patient 105. Although leads 130 may each be a single lead, a lead 130 may include a lead extension or other segments that may aid in implantation or positioning of lead 130.

The electrodes 132 of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration. Deployment of electrodes via leads 130 is described for purposes of illustration, but electrodes may be arranged on a housing of IMD 110, e.g., in rows and/or columns (or other arrays or patterns), as surface electrodes, ring electrodes, or protrusions.

Neurostimulation stimulation parameters defining the electrical stimulation pulses delivered by IMD 110 through electrodes 132 of leads 130 may include information identifying which electrodes have been selected for delivery of the stimulation pulses according to a stimulation program and the polarities of the selected electrodes (the electrode combination), and voltage or current amplitude, pulse rate (i.e., frequency), and pulse width of the stimulation pulses. The neurostimulation stimulation parameters may further include a cycling parameter that specifies when, or how long, stimulation is turned on and off. Neurostimulation stimulation parameters may be programmed prior to delivery of the neurostimulation pulses, manually adjusted based on user input, or automatically controlled during delivery of the neurostimulation pulses, e.g., based on sensed conditions.

Although the example of FIG. 1 is directed to SCS therapy, e.g., to treat pain, in other examples, system 100 may be configured to treat other conditions that may benefit from neurostimulation therapy. For example, system 100 may be used to treat tremor, Parkinson's disease, epilepsy, or other neurological disorders, urinary or fecal incontinence, sexual dysfunction, obesity, or gastroparesis, or psychiatric disorders such as depression, mania, obsessive compulsive disorder, or anxiety disorders. Hence, in some examples, system 100 may be configured to deliver sacral neuromodulation (SNM), deep brain stimulation (DBS), peripheral nerve stimulation (PNS), or other stimulation, such as peripheral nerve field stimulation (PNFS), cortical stimulation (CS), gastrointestinal stimulation, or any other stimulation therapy capable of treating a condition of patient 105. In some examples, system 100 may be configured where the electrical stimulation includes stimulation parameters to deliver therapy to address a condition of one or more of painful diabetic neuropathy (PDN), peripheral vascular disease (PVD), peripheral artery disease (PAD), complex regional pain syndrome (CRPS), angina pectoris (AP), leg pain, back pain or pelvic pain.

Leads 130 may include, in some examples, one or more sensors configured to sense one or more physiological stimulation parameters of patient 105, such as patient activity, pressure, temperature, posture, heart rate, or other characteristics. At least some of electrodes 132 may be used to sense electrical signals within patient 105, additionally or alternatively to delivering stimulation. IMD 110 is configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation. In some examples, the target tissue includes nerves, smooth muscle or skeletal muscle. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical or lumbar regions.

Stimulation of spinal cord 120 may, for example, prevent pain signals from being generated and/or traveling through spinal cord 120 and to the brain of patient 105. Patient 105 may perceive the interruption of pain signals as a reduction in pain and, therefore, efficacious therapy results. In some examples, stimulation of spinal cord 120 may produce paresthesia which may reduce the perception of pain by patient 105, and thus, provide efficacious therapy results. In other examples, stimulation of spinal cord 120 may be effective in reducing pain with or without presenting paresthesia. In some examples, some electrical stimulation pulses may be directed to glial cells while other electrical stimulation (e.g., delivered by a different electrode combination and/or with different stimulation parameters) is directed to neurons. In other examples, stimulation of spinal cord 120 may be effective in promoting blood flow in one or more remote tissue locations, e.g., in a limb or appendage, thereby alleviating or reducing pain or other symptoms, or preventing or delaying onset of tissue damage or degeneration.

IMD 110 generates and delivers electrical stimulation therapy to a target stimulation site within patient 105 via the electrodes of leads 130 to patient 105 according to one or more therapy stimulation programs. A therapy stimulation program specifies values for one or more stimulation parameters that define an aspect of the therapy delivered by IMD 110 according to that program. For example, a stimulation therapy program that controls delivery of stimulation by IMD 110 in the form of stimulation pulses may define values for voltage or current pulse amplitude, pulse width, and pulse rate (e.g., pulse frequency) for stimulation pulses delivered by IMD 110 according to that program, as well as the particular electrodes and electrode polarities forming an electrode combination used to deliver the stimulation pulses. Hence, a stimulation therapy program may specify the location(s) at which stimulation is delivered and amplitude, pulse width and pulse rate of the stimulation. In some examples, a stimulation therapy program may additionally specify cycling of the stimulation, e.g., in terms of that when, or how long, stimulation is turned on and off.

A user, such as a clinician or patient 105, may interact with a user interface of an external programmer 150 to program IMD 110. Programming of IMD 110 may refer generally to the generation and transfer of commands, programs, or other information to control the operation of IMD 110. In this manner, IMD 110 may receive the transferred commands and programs from external programmer 150 to control electrical stimulation therapy. For example, external programmer 150 may transmit therapy stimulation programs, stimulation parameter adjustments, therapy stimulation program selections, user input, or other information to control the operation of IMD 110, e.g., by wireless telemetry or wired connection.

In some cases, external programmer 150 may be characterized as a physician or clinician programmer if it is primarily intended for use by a physician or clinician. In other cases, external programmer 150 may be characterized as a patient programmer if it is primarily intended for use by a patient. A patient programmer may be generally accessible to patient 105 and, in many cases, may be a portable device that may accompany patient 105 throughout the patient's daily routine, e.g., as a handheld computer similar to a tablet or smartphone. For example, a patient programmer may receive input from patient 105 when the patient wishes to terminate or change stimulation therapy. In general, a physician or clinician programmer may support selection and generation of programs by a clinician for use by IMD 110, and may take the form, for example, of a handheld computer (e.g., a tablet computer), laptop computer or desktop computer, whereas a patient programmer may support adjustment and selection of such programs by a patient during ordinary use. In other examples, external programmer 150 may include, or be part of, an external charging device that recharges a power source of IMD 110. In this manner, a user may program and charge IMD 110 using one device, or multiple devices.

IMD 110 and external programmer 150 may exchange information and may communicate via wireless communication using any techniques known in the art. Examples of communication techniques may include, for example, radiofrequency (RF) telemetry and inductive coupling, but other techniques are also contemplated. In some examples, external programmer 150 includes a communication head that may be placed proximate to the patient's body near the IMD 110 implant site to improve the quality or security of communication between IMD 110 and external programmer 150. Communication between external programmer 150 and IMD 110 may occur during power transmission or separate from power transmission.

IMD 110, in response to commands from external programmer 150, may deliver electrical stimulation therapy according to a plurality of therapy stimulation programs to a target tissue site of the spinal cord 120 of patient 105 via electrodes 132 on leads 130. In some examples, IMD 110 automatically modifies therapy stimulation programs as therapy needs of patient 105 evolve over time. For example, the modification of the therapy stimulation programs may cause the adjustment of at least one parameter of the plurality of stimulation pulses based on received information.

IMD 110 and/or external programmer 150 may receive information from one or more blood flow sensors 160, e.g., directly via wireless communication or indirectly from an intermediate server via a network connection. Blood flow sensor 160 may be positioned to sense blood flow at a selected location on patient 105. In some examples, a blood flow sensor 160 may be positioned at, attached to or near tissue for a target anatomical area, e.g., at a limb or appendage, such as at or on a leg, toe, foot, arm, finger or hand of patient 105, to sense blood flow in the tissue adjacent to placement of the blood flow sensor 160. In some examples, a blood flow sensor 160 may be attached to an appendage of the patient 105 to sense the blood flow associated with the appendage, e.g., by a clip-on mechanism, strap, elastic band and/or adhesive. In some examples, blood flow sensor 160 (or one of a plurality of blood flow sensors) may be implantable within patient 105, e.g., within a limb or appendage of the patient.

The blood flow sensor 160 measures blood flow and provides information related to blood flow associated with tissue of the patient. For example, the blood flow sensor 160 may provide blood flow values, or other information indicative of blood flow values or changes in blood flow values, for tissue at the selected location of the patient. The blood flow value may be an instantaneous blood flow measurement, or may be a measurement of blood flow over a period of time such as average blood flow value, maximum blood flow value, minimum blood flow value during the period of time. The IMD 110 provides therapy using stimulation having a particular set of stimulation parameters, and the programmer 150 may be used to modify the stimulation parameters for delivery of the stimulation. Using the information related to the blood flow associated with the tissue of the patient, for example blood flow values, the particular stimulation parameters for therapy delivered by the IMD 110 may be selected or adjusted.

The blood flow sensor 160 may be used to determine whether a blood flow value or range of blood flow values has been achieved for a set of stimulation parameters of the IMD. For example, one or more processors of blood flow sensor 160, programmer 150 or IMD 110 may be configured to determine whether electrical stimulation with a particular set of stimulation parameters resulted in a sensed blood flow value or change in sensed blood flow value that was above a predetermined level, below a predetermined level, and/or within a range prescribed by upper and lower levels. The blood flow sensor 160 may provide information on whether there is a change in blood flow value for the area targeted by delivery of stimulation by the IMD 110 or changes in delivery of stimulation by the IMD 110 (e.g., an area of tissue in a limb or appendage targeted by spinal cord stimulation for a change in blood flow). In some examples, the blood flow sensor 160 may send raw blood flow information or a change in blood flow values to the external programmer 150, and the programmer 150 displays the raw blood flow information or change in blood flow values. The blood flow values may be reviewed manually by a clinician or automatically evaluated by one or more processors of blood flow sensor 160, external programmer 150 and/or IMD 110, or other remote processors via network connection.

Evaluation of the blood flow value, including changes in blood flow value or blood flow value as compared to levels, ranges, or a matrix of predetermined blood flow values as compared to a base line blood flow value, in conjunction with the stimulation parameters, may indicate efficacy of stimulation parameters, such as location of stimulation (e.g., by selection of particular electrode combination) or the way the stimulation is delivered (e.g., by selection of different amplitude, pulse width, pulse rate, and/or duty cycle). If the desired blood flow value or change in blood flow value is not detected, the programmer 150 can be used, for example via a user interface, to change the stimulation parameters, and the revised stimulation parameters may be objectively evaluated by reviewing the blood flow values obtained while the IMD 110 provides stimulation using the revised stimulation parameters. The stimulation parameters may be manually changed for example in a clinical setting, e.g., via external programmer 150, remotely by a clinician, e.g., via a web browser client or application running on a remote computer, or automatically changed in a closed loop system, e.g., by external programmer 150 or IMD 110. The revised stimulation parameters may be evaluated as producing either an improvement or decrease of the effectiveness of the stimulation therapy using the blood flow values. By setting and adjusting stimulation parameters including electrode location using the blood flow information, the system 100 and IMD 110 may be configured to deliver objectively efficacious therapy results for one or more diseases or disorders, such as painful diabetic neuropathy (PDN), peripheral vascular disease (PVD), peripheral artery disease (PAD), complex regional pain syndrome (CRPS), angina pectoris (AP), leg pain, back pain or pelvic pain, e.g., alone or in addition to pain typically addressed by SCS.

FIGS. 2A and 2B are block diagrams illustrating example configurations of components of an IMD 200, in accordance with one or more techniques of this disclosure. IMD 200 may be an example of IMD 110 of FIG. 1. IMD 200 includes stimulation generation circuitry 202, switch circuitry 204, sensing circuitry 206, telemetry circuitry 208, processing circuitry 210, storage device 212, sensor(s) 222, power source 224, lead 230A carrying electrodes 232A, which may correspond to lead 130A and electrodes 132A of FIG. 1, and lead 230B carrying electrodes 232B, which may correspond to lead 130B and electrodes 132B of FIG. 1. Processing circuitry 210 may include one or more processors configured to perform various operations of IMD 200. In the examples shown in FIGS. 2A and 2B, IMD 200 includes stimulation generation circuitry 202, switch circuitry 204, sensing circuitry 206, telemetry circuitry 208, processing circuitry 210, sensor(s) 222, power source 224, lead 230A carrying electrodes 232A, which may correspond to lead 130A and electrodes 132A of FIG. 1, and lead 230B carrying electrodes 232B, which may correspond to lead 130B and electrodes 132B of FIG. 1.

In the examples shown in FIGS. 2A and 2B, storage device 212 stores stimulation parameter settings 242. In addition, as shown in FIG. 2A, storage device 212 may store blood flow data 254 obtained directly or indirectly from one or more blood flow sensors 160 (FIG. 1), and a blood flow correlation index 252 that defines correlations between blood flow information and parameter information for delivery of electrical stimulation for neurostimulation, e.g., by indexing stimulation parameters or parameter adjustments to blood flow value indicating blood flow values or changes in blood flow value. In this case, IMD 200 of FIG. 2A may process sensed blood flow information and select or adjust stimulation parameter settings based on the blood flow information, or the processor circuitry of the IMD 200 automatically adjusts one or more of the stimulation parameters based on the relationship defined by the correlation index. In one or more examples, the parameter information may include one or more electrical stimulation parameters or parameter adjustments. In some examples, the blood flow information includes a differential between sensed blood flow values and target blood flow values, and the parameter information includes electrical stimulation parameter adjustments.

In one or more examples, the IMD 200 does not store or receive the sensed blood flow information, as shown in FIG. 2B. Instead, external programmer 150 or another device may directly or indirectly select or adjust stimulation parameter settings based on sensed blood flow information and communicate the selected settings or adjustments to IMD 200 of FIG. 2A. In some examples, stimulation parameter settings 242 may include stimulation parameters (sometimes referred to as “sets of therapy stimulation parameters”) for respective different stimulation programs selectable by the clinician or patient for therapy. In some examples, stimulation parameter settings 242 may include one or more recommended parameter settings. IMD 200 may be programmed to deliver subsequent electrical stimulation using at least some of the one or more recommended parameter settings. In this manner, each stored therapy stimulation program, or set of stimulation parameters, of stimulation parameter settings 242 defines values for a set of electrical stimulation parameters (e.g., a stimulation parameter set), such as electrode combination (selected electrodes and polarities), stimulation current or voltage amplitude, stimulation pulse width, pulse rate, or duty cycle. In some examples, stimulation parameter settings 242 may further include cycling information indicating when or how long stimulation is turned on and off, (i.e., duty cycling). For example, recommended parameter settings may indicate the stimulation to turn on for a certain period of time, and/or to turn off stimulation for a certain period of time. In another example, recommended duty cycle parameter settings may indicate stimulation to turn on for a period of time without creating desensitization of the stimulation. In one or more examples, the recommended parameter settings may indicate stimulation to occur at a certain time of day, for example when the patient is typically awake or active, or sleeping. In one or more examples, recommended parameter settings relate to when the patient has a certain posture, for example when the patient is in a supine position.

Stimulation generation circuitry 202 includes electrical stimulation circuitry configured to generate electrical stimulation and generates electrical stimulation pulses selected to alleviate symptoms of one or more diseases, disorders or syndromes. While stimulation pulses are described, stimulation signals may take other forms, such as continuous-time signals (e.g., sine waves) or the like. The electrical stimulation circuitry may reside in an implantable housing, for example of the IMD. Each of leads 230A, 230B may include any number of electrodes 232A, 232B. The electrodes are configured to deliver the electrical stimulation to the patient. In the example of FIGS. 2A and 2B, each set of electrodes 232A, 232B includes eight electrodes A-H. In some examples, the electrodes are arranged in bipolar combinations. A bipolar electrode combination may use electrodes carried by the same lead 230A, 230B or different leads. For example, an electrode A of electrodes 232A may be a cathode and an electrode B of electrodes 232A may be an anode, forming a bipolar combination. Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 232A, 232B, or directed sensed signals from one or more of electrodes 232A, 232B to sensing circuitry 206. In some examples, each of the electrodes 232A, 232B may be associated with respective regulated current source and sink circuitry to selectively and independently configure the electrode to be a regulated cathode or anode. Stimulation generation circuitry 202 and/or sensing circuitry 206 also may include sensing circuitry to direct electrical signals sensed at one or more of electrodes 232A, 232B.

Sensing circuitry 206 may be configured to monitor signals from any combination of electrodes 232A, 232B. In some examples, sensing circuitry 206 includes one or more amplifiers, filters, and analog-to-digital converters. Sensing circuitry 206 may be used to sense physiological signals, such as evoked compound action potential (ECAP) signals. In some examples, sensing circuitry 206 detects ECAP signals from a particular combination of electrodes 232A, 232B. In some cases, the particular combination of electrodes for sensing ECAP signals includes different electrodes than a set of electrodes 232A, 232B used to deliver stimulation pulses. Alternatively, in other cases, the particular combination of electrodes used for sensing ECAP signals includes at least one of the same electrodes as a set of electrodes used to deliver stimulation pulses to patient 105. Sensing circuitry 206 may provide signals to an analog-to-digital converter, for conversion into a digital signal for processing, analysis, storage, or output by processing circuitry 210.

Telemetry circuitry 208 supports wireless communication between IMD 200 and an external programmer, blood flow sensing system, or another computing device under the control of processing circuitry 210. Processing circuitry 210 of IMD 200 may receive, as updates to programs, values for various stimulation parameters such as amplitude and electrode combination, from the external programmer via telemetry circuitry 208. Processing circuitry 210 may store updates to the stimulation parameter settings 242 or any other data in storage device 212. Telemetry circuitry 208 in IMD 200, as well as telemetry circuits in other devices and systems described herein, such as the external programmer and blood flow sensing system, may accomplish communication by radiofrequency (RF) communication techniques. In addition, telemetry circuitry 208 may communicate with an external medical device programmer via proximal inductive interaction of IMD 200 with the external programmer, where the external programmer may be one example of external programmer 150 of FIG. 1. Accordingly, telemetry circuitry 208 may send information to the external programmer or the blood flow sensing system on a continuous basis, at periodic intervals, or upon request from IMD 110 or the external programmer.

Processing circuitry 210 may include one or more processors, such as any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 controls stimulation generation circuitry 202 to generate stimulation signals according to stimulation parameter settings 242. In some examples, processing circuitry 210 may execute other instructions stored in storage device 212 to apply stimulation parameters specified by one or more of programs, such as amplitude, pulse width, pulse rate, and pulse shape of each of the stimulation signals.

In the illustrated example of FIG. 2A, processing circuitry 210 includes a blood flow unit 216 to process the blood flow information. Blood flow unit 216 may represent an example of a portion of processing circuitry configured to process blood flow information received from a blood flow sensor, such as blood flow sensor 160. In the example of FIG. 2B, the processing of blood flow information occurs in a device other than IMD 200. Referring again to FIG. 2A, the blood flow unit 216, discussed further below, receives information regarding the blood flow data, such as information relating to sensed blood flow associated with tissue of the patient 105, and controls the electrical stimulation circuitry 202 to deliver the electrical stimulation to the patient based on the received information, where the indications of the received information may be stored in a storage device. Blood flow unit 216 may select or adjust electrical stimulation parameter settings in response to sensed blood flow, e.g., to maintain blood flow within, or drive blood flow into, a desired range, or above a predetermined level, or below a predetermined level, where the range or level may be selected to promote beneficial levels of blood flow to alleviate, reduce or delay onset of symptoms of diseases or disorders, or delay onset of damage or degeneration of tissue. In one example, IMD 200 delivers spinal cord stimulation to a first tissue site associated with the spine of patient 105 with one or more parameter settings selected or adjusted based on blood flow sensed in a second tissue site associated with a second tissue site, e.g., in a limb or appendage, such as a foot, toe, hand, or finger, remote from the first tissue site, to promote desired levels of blood flow within the limb or appendage. Processing circuitry 210 also controls stimulation generation circuitry 202 to generate and apply the stimulation signals to selected combinations of electrodes 232A, 232B. In some examples, stimulation generation circuitry 202 includes a switch circuit (instead of, or in addition to, switch circuitry 204) that may couple stimulation signals to selected conductors within leads 230, which, in turn, deliver the stimulation signals across selected electrodes 232A, 232B. Such a switch circuit may selectively couple stimulation energy to selected electrodes 232A, 232B and to selectively sense bioelectrical neural signals of a spinal cord of the patient with selected electrodes 232A, 232B. In other examples, however, stimulation generation circuitry 202 does not include a switch circuit and switch circuitry 204 does not interface between stimulation generation circuitry 202 and electrodes 232A, 232B. In these examples, stimulation generation circuitry 202 may include a plurality of pairs of current sources and current sinks, each connected to a respective electrode of electrodes 232A, 232B. In other words, in these examples, each of electrodes 232A, 232B is independently controlled via its own stimulation circuit (e.g., via a combination of a regulated current source and sink), as opposed to switching stimulation signals between different electrodes of electrodes 232A, 232B.

Storage device 212 may be configured to store information within IMD 200 during operation. Storage device 212 may include a computer-readable storage medium or computer-readable storage device. In some examples, storage device 212 includes one or more of a short-term memory or a long-term memory. Storage device 212 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions, e.g., for execution by processing circuitry 210. As discussed above, storage device 212 is configured to store stimulation parameter settings 242.

Power source 224 is configured to deliver operating power to the components of IMD 200. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200. Power source 224 may include any one or more of a plurality of different battery types, such as nickel cadmium batteries and lithium ion batteries.

In some examples as shown in FIG. 2A, the processing circuitry 210 of the IMD 200 directs delivery of electrical stimulation by the electrodes 232A, 232B of leads 230A, 230B, receives information relating to blood flow from the blood flow sensor, and generates output based on the received information. The blood flow unit 216 may use blood flow information to develop efficacy indications or recommended electrical stimulation parameters or adjustments which are outputted to a user, where the user can use the indications or one or more recommended stimulation parameters to program the IMD 200, e.g., by selecting or accepting the recommendations as stimulation parameter settings to be used by IMD 200. For example, a particular electrode combination is recommended to a user and/or a set of stimulation parameters are recommended to a user and presented to the user via the programmer. The user may accept the recommended electrode combination and/or one or more recommended stimulation parameters, and the programmer programs IMD 200 to implement and deliver stimulation with the selected electrode combination and/or stimulation parameters.

In some examples, efficacy may be determined by delivering electrical stimulation with differing combinations of stimulation parameters, and monitoring sensed blood flow and changes in sensed blood flow as a result of delivery of electrical stimulation according to the differing stimulation parameters. The stimulation parameters may include, but are not limited to, electrode combination (e.g., selected electrodes and polarities), stimulation amplitude, stimulation pulse width, stimulation frequency, or duty cycle. Changes in blood flow value may be changes from a measured baseline blood flow value when stimulation was not delivered, or a previous blood flow value when stimulation was delivered with particular stimulation parameters. In one or more examples, the blood flow device may be calibrated by detecting blood flow with and without stimulation.

Processing circuitry 210 controls stimulation circuitry 202 to deliver stimulation energy with stimulation parameters specified by one or more stimulation parameter settings 242 stored on storage device 212 and, in the example of FIG. 2A, to collect blood flow information pertaining to the stored stimulation parameter settings 242. Processing circuitry 210 collects this blood flow information by receiving the information via telemetry from a remote blood flow sensor at a remote site. Other options for the IMD 200 include the processing circuitry 210 collecting the flood flow information from an onboard sensor for local blood flow sensing, such as sensing blood flow at the spinal tissue site. Processing circuitry 210 may also control stimulation circuitry 202 to test different parameter settings and record corresponding blood flow values for each selected combination, and test different parameter settings as they compare to sensed blood flow. For example, processing circuitry 210 directs stimulation circuitry 202 to deliver stimulation via a particular electrode combination and/or with a particular amplitude (and other parameter settings) and the blood flow unit 216 collects the corresponding blood flow value from telemetry circuitry 208. The blood flow data 254 for this test may be stored in the storage device 212. Processing circuitry 210 may adjust the previously tested amplitude value of the stimulation delivered via the electrode combination to a different value and collect the corresponding blood flow value from the blood flow sensor in response to stimulation with the adjusted amplitude. The blood flow value received for the stimulation at the changed stimulation parameter, in this example, amplitude, would be saved in the storage device 212 and may be output to a user. The processing circuitry 210 may continue to shift the amplitude values by either increasing or decreasing the amplitude, and recording the respective blood flow values, which are stored on the storage device 212 and the information may be output to a user. While the example of amplitude is provided, processing circuitry 210 may direct stimulation circuitry 202 to step through various incremental settings of other stimulation parameters, such as stimulation pulse width, stimulation frequency, or duty cycle, and record the respective blood flow information for each stepped value. In one or more examples, processing circuitry 210 may direct stimulation circuitry to turn on for a certain period of time, and/or to turn off for a period of time, or to turn on at a certain time of day and record the respective blood flow value. In one example, the processing circuitry 210 cycles stimulation to turn on for 12 hours, and turn off for 12 hours. Stimulation circuitry 202 may shift more than one stimulation parameter for each test and collect sensed blood flow information for each of the multiple shifted stimulation parameters.

Blood flow unit 216 processes the collected sensed blood flow information. In some examples, blood flow unit 216 communicates the blood flow information to a user and is configured to output the blood flow information, where the output includes one or more blood flow values. The user can use the blood flow information to determine efficacy of a particular stimulation parameter setting, or a group of stimulation parameters, or track trends in blood flow changes over time, e.g., with and without electrical stimulation, or with different parameter settings.

In some examples, the blood flow unit 216 processes the information to perform closed loop control of the stimulation parameters based on the blood flow information. The blood flow unit 216 may store the blood flow data 254 in storage device 212, and may interact with and/or develop a blood flow correlation index to automatically adjust stimulation parameter settings 242 based on the blood flow information. For example, blood flow unit 216 may select or adjust one or more settings of parameter values, such as electrode combination, amplitude, pulse width or pulse rate, in response to sensed blood flow information. The blood flow information may be sensed when electrical stimulation is not delivered or upon delivery of electrical stimulation. In either case, blood flow unit 216 may be configured to direct or control stimulation circuitry 202 to select or adjust one or more settings of parameter values to cause the sensed blood to be above a predetermined level, below a predetermined level, or within a desired range of blood flow values known or expected to be beneficial to patient 105.

In some examples of determining efficacy of lead placement, selecting stimulation parameters includes a series of testing of electrode combinations, e.g., at different positions, may occur so that a user may identify desirable combinations or the processing circuitry 210 can develop recommended electrode combinations, e.g., for acceptance or selection by a user, based at least in part by the blood flow information. For example, a recommended electrode combination may be based on an electrode combination that achieves the most desirable blood flow reading during stimulation, or a blood flow that is above a predetermined level, below a predetermined level, or within a desired range of blood flow values known or expected to be beneficial to patient 105. Processing circuitry 210 causes the IMD to scan though each of a plurality of electrode combinations (e.g., in a sequence that includes different positions within a tissue site, such as different positions along the length of one or more leads implanted adjacent the spinal cord for spinal cord stimulation), and for each combination, a sensed blood flow value is recorded for the particular electrode combination. In this example, processing circuitry 210 shifts stimulation energy between different electrode combinations, and monitors changes in blood flow as a result of the different electrode combinations. Processing circuitry 210 may also control stimulation circuitry 202 to deliver, for each electrode combination, stimulation with different stimulation parameter combinations (e.g., amplitude, pulse width pulse rate, and/or duty cycle) and the sensed blood flow value is recorded for the particular electrode combination and stimulation parameter combination. The changes in electrode combinations and/or parameter combinations may be manually changed by a user, or processing circuitry 210 may automatically test the various electrode pairs to, in effect scan through different positions and stimulation parameters of the electrical stimulation, and record the corresponding blood flow information.

As an illustration for electrode pairing for the blood flow testing, IMD 200 may be coupled to a 2×8 electrode arrangement in which two leads each carry eight electrodes, and the electrodes on one lead are designated 0 through 7 from top to bottom, and the electrodes on the other lead are designated 8-15 from top to bottom. In this example, a first electrode combination could be the following: 0+1−2+, where the number designates the electrode position and the plus or minus designates the polarity of the electrode.

In this example, a shift to a second electrode combination could yield the same pattern but simply move down one electrode position, e.g., 1+2−3+. In other embodiments, the first and second electrode combinations may have different patterns, e.g., combination 1=0+1− and combination 2=0+1−2+, and then combination 3=1+2−. Combinations between the leads (e.g., 1-7) could also be tested. For each combination of electrode pairing, different parameter settings could be tested, e.g., for electrodes 0-1, a set of different amplitudes, different pulse widths, or different frequencies, or combinations thereof. In one or more examples, unipolar combinations are paired and tested, with a single electrode 16 on the lead and an electrode on IMD housing (e.g., 0-16, 1-16, 2-16 . . . ). In one or more examples, multipolar combinations are tested, with three of more electrodes in various patterns (for example, 0-1-7, where 0 and 7 are cathodes and 1 is an anode).

For each of the electrode combinations and parameter combinations, the corresponding blood flow value is sensed and stored, where the sensed and stored blood value is recorded as being associated with the particular electrode combination and parameter combination. Upon scanning a plurality of electrode combinations and stimulation parameter combinations, storage device 212 may store a plurality of associated blood flow values, e.g., for review by a clinician and/or for use in directing or control delivery of stimulation by IMD 200.

Blood flow unit 216 receives blood flow data 254 to store in storage device 212. The blood flow data 254 may be raw data from the blood flow sensor 160 such as blood flow, blood flow change, rate of change of blood flow, or processed data. The blood flow data may include instantaneous blood flow measurements, or may be a measurement of blood flow over a period of time such as average blood flow value, maximum blood flow value, minimum blood flow value during the period of time. Blood flow data may include data from the incrementally tested stimulation parameters discussed above. The processed data may include raw blood flow data that has been evaluated and processed into other categories, such as a rating of high, medium, low change relative to a baseline blood flow value. In some examples, the processed data relates to a numeric score, or a value rating, indicating a relative level of blood flow.

The blood flow unit 216 may use the blood flow data 254 to interact with and/or develop a blood flow correlation index 252. The blood flow correlation index 252 may include a matrix of information that tracks a relationship between two or more variables. For example, a first set of stimulation parameters (e.g., electrode combination, amplitude, pulse width, pulse rate, and/or duty cycle) may result in a first blood flow value, and a second set of stimulation parameters may result in a second blood flow value. In one or more examples, a third set of stimulation parameters may result in a third blood flow value.

The first and second blood flow values, and optionally the third blood flow value, achieved using the first, second and third set of stimulation parameters may be further categorized within the blood flow correlation index 252 by additional factors such as factors dependent on the patient condition, such as patient activity level, patient posture, sensed glucose level, sensed patient temperature, sensed patient heart rate, patient diet input, patient pain input, patient sensitivity input, and/or other input from patient sensors. Additional factors can include factors independent of the patient including time of day, temperature, or increments of time. The correlation index 252 may include a log of blood flow over time, and also after stimulation parameter settings have been adjusted. In one or more examples, the correlation index may include an input of a target blood flow value or target blood flow value change to be achieved, and as output a set of stimulation parameters or adjustments to produce the target blood flow or target blood flow change. In one or more examples, the inputs may further include patient condition, such as patient activity level, patient posture, sensed glucose level, patient diet input, patient pain input, patient sensitivity input, and/or other input from patient sensors. Additional inputs may include factors independent of the patient including time of day, temperature, or increments of time. In some examples, the index maps the target blood flow value or target blood flow value change to a set of stimulation parameters necessary to produce the target blood flow value or target blood flow value change. In some examples, the index maps the target blood flow value or target blood flow value change to a patient condition necessary to produce the target blood flow value or target blood flow value change. In an example, the index maps the target blood flow value or target blood flow value change to a patient activity level, patient posture, sensed glucose level, patient diet input, patient pain input, patient sensitivity input, and/or other input from patient sensors necessary to produce the target blood flow value or target blood flow value change. In some examples, the index maps the target blood flow value or target blood flow value change to a time of day, temperature, or increments of time necessary to produce the target blood flow value or target blood flow value change.

The blood flow unit 216 may use the blood flow data 254 with or without the blood flow correlation index to inform a user of recommended parameter settings or automatically adjust stimulation parameter settings 242 using the IMD 200. For example, the IMD 200 may incrementally adjust stimulation parameters up or down in fixed increments until a target blood flow value is achieved. Again, selected or adjusted stimulation parameter settings 242 may include electrode combinations (and hence location of stimulation), stimulation amplitude, stimulation pulse width, stimulation pulse rate, and/or duty cycle, and may further include consideration of patient activity level, patient posture, sensed glucose level, patient diet input, patient pain input, patient sensitivity input, other input from patient sensors, patient temperature, external temperature, and/or time of day. The blood flow unit 216 receives the blood flow data 254, and the blood flow unit 216 processes the data to determine if stimulation should be adjusted, for example, if the blood flow data 254 falls below a threshold blood flow value, increases beyond an upper limit for blood flow value, or falls outside of a range of values. In an example, blood flow unit 216 may select stimulation parameters or apply a prescribed adjustment to stimulation parameters. If blood flow unit 216 determines the stimulation parameters should be changed based on the current or trending blood flow values, the blood flow unit 216 may automatically implement a change in one or more stimulation parameter settings and record the revised blood flow data for the adjusted stimulation parameter settings, or blood flow unit 216 may recommend a change in parameter settings to a user, for example by communication to an external controller. If the implemented changes in the one or more stimulation parameters settings do not achieve an expected or desired blood flow, the stimulation parameter settings may be changed, and the blood flow value is evaluated again. This process may be repeated until the desired blood flow value is achieved.

In some examples, the processing circuitry 210 of the IMD 200 directs delivery of electrical stimulation of the electrodes 232A, 232B, and receives information relating to blood flow from one or more blood flow sensors 160, either directly or via external controller, and controls the delivery of electrical stimulation of the electrodes 232A, 232B based on the received information in a closed loop setting. The blood flow information may be received via the telemetry circuitry 208 either directly or indirectly from the blood flow sensor 160 (FIG. 1). In an example, the IMD 200 may receive the blood flow information from an intermediate device other than the blood flow sensor, such as external programmer 150.

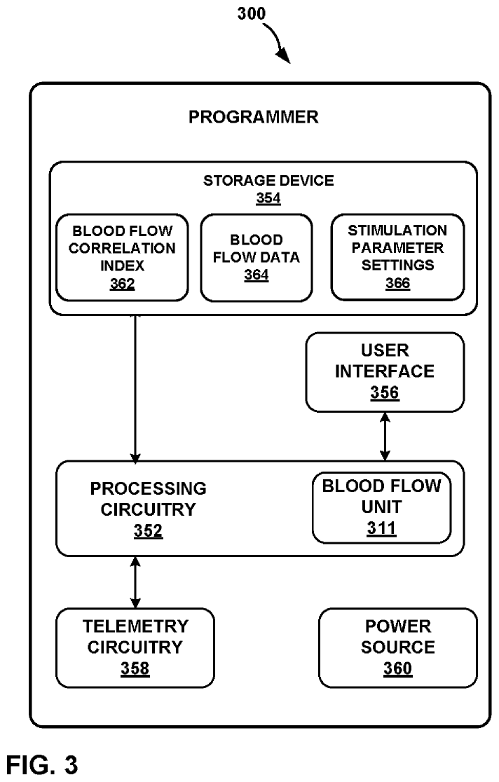

In some examples, as shown in FIG. 2B, the processing circuitry 210 of IMD 200 directs delivery of electrical stimulation of the electrodes 232A, 232B based on receiving adjustments from an external or remote controller (FIG. 3). In one or more examples, the external or remote controller, such as a programmer, receives information relating to blood flow associated with tissue upon the delivery of the electrical stimulation, and processes the blood flow information as discussed below. The processing circuitry 210 of the IMD 200 is configured to receive adjustments to the stimulation parameters from the external programmer based on the blood flow information, and to direct delivery of electrical stimulation of the adjusted stimulation parameters.

FIG. 3 is a block diagram illustrating an example configuration of components of an example external programmer 300. External programmer 300 may be an example of external programmer 150 of FIG. 1. Although external programmer 300 may generally be described as a hand-held device, such as a tablet computer or smartphone-like device, external programmer 300 may be a larger portable device, such as a laptop computer, or a more stationary device, such as a desktop computer. In addition, in other examples, external programmer 300 may be included as part of an external charging device or include the functionality of an external charging device, e.g., to recharge a battery or batteries associated with IMD 200. As illustrated in FIG. 3, external programmer 300 may include processing circuitry 352, storage device 354, user interface 356, telemetry circuitry 358, and power source 360. In some examples, storage device 354 may store instructions that, when executed by processing circuitry 352, cause processing circuitry 352 and external programmer 300 to provide the functionality ascribed to external programmer 300 throughout this disclosure. Each of these components, circuitry, or modules, may include electrical circuitry that is configured to perform some, or all of the functionality described herein. For example, processing circuitry 352 may include processing circuitry configured to perform the processes discussed with respect to processing circuitry 352.

In general, external programmer 300 includes any suitable arrangement of hardware, alone or in combination with software and/or firmware, to perform the techniques attributed to external programmer 300, and processing circuitry 352, user interface 356, and telemetry circuitry 358 of external programmer 300. In various examples, processing circuitry 352, telemetry circuitry 358, or other circuitry of external programmer 300 may include one or more processors, such as one or more microprocessors, DSPs, ASICs, FPGAs, or any other equivalent integrated or discrete logic circuitry, as well as any combinations of such components. External programmer 300 also, in various examples, may include a storage device 354, such as RAM, ROM, PROM, EPROM, EEPROM, flash memory, a hard disk, a CD-ROM, including executable instructions for causing the one or more processors to perform the actions attributed to them. Moreover, although processing circuitry 352 and telemetry circuitry 358 are described as separate modules, in some examples, processing circuitry 352 and telemetry circuitry 358 are functionally integrated. In some examples, processing circuitry 352, telemetry circuitry 358 or other circuitry of external programmer 300 may correspond to individual hardware units, such as ASICs, DSPs, FPGAs, or other hardware units.

The processing circuitry 352 is configured to direct delivery of electrical stimulation, receive information relating to blood flow associated with tissue upon the delivery of the electrical stimulation, and generate as at least part of the output based on the received information, e.g., for evaluation of efficacy of stimulation parameters and/or recommend, or assist a user in programming, stimulation parameters for delivery of electrical stimulation. In some examples, the processing circuitry 352 is configured to control the electrical stimulation circuitry to deliver the electrical stimulation based on the blood flow information in a closed loop basis by directing the IMD to use particular stimulation parameters.

Storage device 354 (e.g., a storage device) may, in some examples, store instructions that, when executed by processing circuitry 352, cause processing circuitry 352 and external programmer 300 to provide the functionality ascribed to external programmer 300 throughout this disclosure. For example, storage device 354 may include instructions that cause processing circuitry 352 to obtain a parameter set from memory or receive user input and send a corresponding command to IMD 200, or instructions for any other functionality. In addition, storage device 354 may include a plurality of programs, where each program includes a parameter set that defines therapy stimulation or control stimulation. Storage device 354 may also store data received from a medical device (e.g., IMD 110) and/or a remote sensing device. For example, storage device 354 may store data recorded at a sensing module of the medical device, and storage device 354 may also store data from one or more sensors of the medical device. In an example, storage device 354 may store data recorded at a remote sensing device such as blood flow values sensed from blood flow sensors.

User interface 356 may include a button or keypad, lights, a speaker for voice commands, a display, such as a liquid crystal (LCD), light-emitting diode (LED), or organic light-emitting diode (OLED). In some examples, the display includes a touch screen. User interface 356 may be configured to display any information related to the delivery of electrical stimulation including output, for example, based on the blood flow information. User interface 356 may also receive user input (e.g., indication of when the patient perceives stimulation, or a pain score perceived by the patient upon delivery of stimulation) via user interface 356. The user input may be, for example, in the form of pressing a button on a keypad or selecting an icon from a touch screen. The input may request starting or stopping electrical stimulation, the input may request a new electrode combination or a change to an existing electrode combination, or the input may request some other change to the delivery of electrical stimulation, such as a change in stimulation amplitude, pulse width or pulse rate.

Telemetry circuitry 358 may support wireless communication between the medical device and external programmer 300 under the control of processing circuitry 352. Telemetry circuitry 358 may also be configured to communicate with another computing device via wireless communication techniques, or direct communication through a wired connection. In some examples, telemetry circuitry 358 provides wireless communication via an RF or proximal inductive medium. In some examples, telemetry circuitry 358 includes an antenna, which may take on a variety of forms, such as an internal or external antenna.

Examples of local wireless communication techniques that may be employed to facilitate communication between external programmer 300 and IMD 110 include RF communication according to the 802.11 or Bluetooth® specification sets or other standard or proprietary telemetry protocols. In this manner, other external devices may be capable of communicating with external programmer 300 without needing to establish a secure wireless connection. As described herein, telemetry circuitry 358 may be configured to transmit a spatial electrode movement pattern or other stimulation parameters to IMD 110 for delivery of electrical stimulation therapy.

Power source 360 is configured to deliver operating power to the components of external programmer 300. Power source 360 may include a battery and a power generation circuit to produce the operating power. In some examples, the battery is rechargeable to allow extended operation. Recharging may be accomplished by electrically coupling power source 360 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium-ion batteries) may be used. In addition, external programmer 300 may be directly coupled to an alternating current outlet to operate.

In some examples, the external programmer 300 or external control device directs delivery of electrical stimulation of an IMD, receives information relating to blood flow associated with tissue upon the delivery of the electrical stimulation, and generates output based on the received information, e.g., for evaluation of efficacy of stimulation parameters and/or recommend, assist a user in programming, stimulation parameters for delivery of electrical stimulation, or used as part of a closed loop control device to automatically adjust stimulation parameters using blood flow information. In one or more examples, the control device generates output based on a first received information and a second received information via a user interface device.

Programmer 300 may be a patient programmer or a clinician programmer and receives blood flow information such as blood flow data 364. Programmer 300 receives blood flow information and allows a user to interact with the processing circuitry 352 via user interface 356 in order to identify efficacious parameter settings, such as electrode combinations and/or one or more other stimulation parameters using the blood flow information. Programmer 300 further assists the user in programming a neurostimulation device by using the blood flow information displayed on the user interface 356. In addition, programmer 300 may be used as part of a closed loop control device to automatically adjust stimulation parameters based at least on blood flow information. In some examples, programmer 300 receives blood flow information such as blood flow data 364 from the blood flow device and stores the blood flow data in the storage device 354.

Programmer 300 may be used to determine efficacy of particular parameter settings of the IMD by testing parameter settings and recording blood flow for each parameter setting. Information resulting from the testing may be presented to a user via the user interface 356. Programmer 300 may receive user input via the user interface device following generation of the output based on the first received information and the second received information, selecting one or more stimulation parameters for the delivery of the electrical stimulation. In one or more examples, the programmer 300 may generate a third set of stimulation parameters for delivery of the electrical stimulation based on the user input. In some examples, the programmer 300 compares the first information that was received relating to the first blood flow with the second information relating to the second blood flow, and automatically generates a third set of stimulation parameters for delivery of the electrical stimulation based on the comparison.

In an example, programmer 300 may be used to cause the IMD to automatically scan though a plurality of electrode combinations or parameter combinations. Processing circuitry 352 causes the IMD to automatically scan through each of a plurality of parameter combinations, including electrode combinations and parameter combinations. For each combination, the programmer 300 obtains and records the corresponding sensed blood flow value.

Alternative to or in addition to the automatic scanning process, the user could manually advance scanning through electrode pairs and/or parameter combinations, for example with an arrow button on user interface 356. In some examples, as the user scans through the electrode pairs or parameter combinations to test and record blood flow values for each combination, the user may collect pain information such as a patient pain score indicating the degree of pain relief information from the combination, or a stimulation perception score indicating whether the patient perceives the stimulation, e.g., by verbal interaction with the patient or patient entry of information via a user input device, and enter the pain information into programmer 300 via user interface 356 of the programmer or the user input device.

Processing circuitry 352 controls stimulation circuitry 202 to deliver stimulation energy with stimulation parameters specified by one or more stimulation parameter settings 366 stored on storage device 354, and to collect blood flow information pertaining to the stored stimulation parameter settings 366. Processing circuitry 352 may also control stimulation circuitry 202 to test different parameter settings and record corresponding blood flow values for each selected combination, and test different parameter settings as they compare to sensed blood flow. For example, processing circuitry 352 directs stimulation circuitry 202 to deliver stimulation with a particular amplitude and the blood flow unit 311 collects the corresponding blood flow value from telemetry circuitry 358. The blood flow data 364 for this test may be stored in the storage device 354 and in the blood flow correlation index 362.

Processing circuitry 352 may be configured to shift the previously tested amplitude value to a different value and collect the corresponding blood flow value from the blood flow sensor. The blood flow value received for the stimulation at the changed stimulation parameter, in this example amplitude, would be saved in the storage device 354. The processing circuitry 352 may continue to shift the amplitude values by either increasing or decreasing the amplitude, and record the respective blood flow values, which are stored on the storage device 354 and the information is output, e.g., via user interface 356. While the example of amplitude is provided, processing circuitry 352 may direct stimulation circuitry to step through various incremental settings of other stimulation parameters, such as stimulation pulse width, stimulation frequency, or duty cycle, and record the respective blood flow information for each stepped value. Stimulation circuitry 202 may shift more than one stimulation parameter for each test and collect sensed blood flow information for the multiple shifted stimulation parameters.

In some examples of parameter testing to determine efficacy of lead placement, a series of testing of electrode combinations may occur so that a user may identify desirable combinations or the processing circuitry 352 and can develop recommended electrode combinations based at least in part by the blood flow information. For example, a recommended electrode combination may be based on an electrode combination that achieves the highest blood flow reading during stimulation, or an electrode combination that achieves a blood flow value that falls within a desired range. Processing circuitry 352 causes the IMD to scan though each of a plurality of electrode combinations, and for each combination, a sensed blood flow value is recorded for the particular electrode combination. Processing circuitry 352 shifts stimulation energy between different electrode combinations, and monitors changes in blood flow as a result of the different electrode combinations. Processing circuitry 352 may also add for each electrode combination, a parameter setting and the sensed blood flow value is recorded for the particular electrode combination and parameter setting. The changes in electrode combinations or parameter value settings may be manually changed by a user, or processing circuitry 352 may automatically test the various electrode combinations and record the corresponding blood flow information, for example by automatically directing delivery.

As an illustration for electrode pairing for the blood flow testing, a 2×8 electrode arrangement in which two leads each carry eight electrodes, and the electrodes on one lead are designated 0 through 7 from top to bottom, and the electrodes on the other lead are designated 8-15 from top to bottom, a first combination could be the following: 0+1−2+, where the number designates the electrode position and the plus or minus designates the polarity of the electrode.

In this example, a shift to a second electrode combination could yield the same pattern but simply move down one electrode position, e.g., 1+2−3+. In other embodiments, the first and second electrode combinations may have different patterns, e.g., combination 1=0+1− and combination 2=0+1−2+, and then combination 3=1+2−. Combinations between the leads (e.g., 1-7) could also be tested. For each combination of electrode pairing, different parameter settings could be tested, e.g., for electrodes 0-1, a set of different amplitudes, different pulse widths, or different frequencies, or combinations thereof. In one or more examples, unipolar combinations are paired and tested, where a single electrode 16 on the lead and an electrode on IMD housing (e.g., 0-16, 1-16, 2-16 . . . ). In one or more examples, multipolar combinations are tested, with three of more electrodes in various patterns (for example, 0-1-7, where 0 and 7 are cathodes and 1 is an anode).

For each of the electrode combinations and parameter settings, the corresponding blood flow information is sensed and stored, where the sensed and stored blood flow value is recorded as being associated with the particular electrode combination and parameter setting. The blood flow information may include blood flow value, blood flow change, blood flow change from a baseline blood flow, average blood flow, or blood volume over a time period.

In some examples, the processing circuitry 352 of programmer 300 directs delivery of electrical stimulation of the electrodes 232A, 232B, and receives information relating to blood flow from the blood flow sensor, and controls the delivery of electrical stimulation of the electrodes 232A, 232B based on the received information in a closed loop setting. The blood flow information may be received via the telemetry circuitry 358 either directly or indirectly from the blood flow sensor 160 (FIG. 1). In an example, the controller receives the blood flow information from an intermediate device other than blood flow sensor 160.

The blood flow unit 311 processes the blood flow information. In some examples, the blood flow unit 311 processes the information to perform closed loop control of the stimulation parameters based on the blood flow information. The blood flow unit 311 may store the blood flow data 364 in storage device 354, and may interact with and/or develop a blood flow correlation index to adjust stimulation parameter settings 366, for example automatically adjust the stimulation parameter settings 366.

In an example, the blood flow unit 311 receives blood flow data 364 to store in storage device 354. The blood flow data 364 may be raw data from the blood flow sensor 160 such as blood flow, blood flow change, rate of change of blood flow, or processed data. The processed data may include raw data that has been evaluated and processed into other categories, such as a rating of high, medium, low. In some examples, the processed data relates to a numeric score, or a value rating.

The blood flow unit 311 may use the blood flow data 364 with or without the blood flow correlation index to develop recommended parameter settings or automatically adjust stimulation parameter settings 366 using the programmer 300. The blood flow unit 311 receives the blood flow data 364, and the blood flow unit 311 processes the data to determine if stimulation should be adjusted, for example if the blood flow data 364 falls below a threshold blood flow value, exceeds an upper limit value, or falls outside of a range of values. If blood flow unit 311 determines the stimulation parameters should be changed based on the current or trending blood flow values, the blood flow unit 311 may automatically implement a change in one or more stimulation parameter settings and record the revised blood flow data for the adjusted stimulation parameter settings, or blood flow unit 311 may recommend a change in parameter settings to a user. The change in stimulation parameter settings may be developed using the blood flow correlation index 362. If the implemented changes in the one or more stimulation parameters settings do not achieve an expected or desired blood flow, the stimulation parameter settings may be changed, and the blood flow value is evaluated again. This process may be repeated until the desired blood flow value is achieved.

Programmer 300 presents to the user a list of the electrode combinations with associated blood flow indications, or a list of combined electrode and parameter combinations with associated blood flow indications so that the user can select one of them. Programmer 300 may also highlight recommended combinations based upon predetermined priorities such as maximizing blood flow with best energy efficiency. For example, programmer 300 may highlight or otherwise identify sets of stimulation parameters (e.g., electrode combinations, electrode polarities, stimulation amplitude, stimulation pulse width, stimulation pulse rate, and/or duty cycle) that produce sensed blood flow values that are closest to a predetermined target blood flow value, above a predetermined blood flow value, below a predetermined blood flow value, or within a blood flow value range. The sets of stimulation parameters may be sortable according to blood flow value or proximity to a predetermined blood flow value. As a further example, programmer 300 highlight or otherwise identify sets of stimulation parameters (e.g., electrode combinations, electrode polarities, stimulation amplitude, stimulation pulse width, stimulation pulse rate, and/or duty cycle) that produce sensed blood flow values that are proximate to a predetermined blood flow value, or within a predetermined blood flow value range, and require less energy consumption to achieve such sensed blood flow values, e.g., in terms of energy consumption associated with the stimulation intensity presented by stimulation amplitude, pulse width, pulse rate, and/or duty cycle. In this manner, programmer 300 may facilitate selection of sets of stimulation parameters that promote desired blood flow values and reduce power consumption by IMD 200.

The provided output of sets of stimulation parameter values and resultant blood flow indications may include raw blood flow values, relative scores (1-x) for blood flow, or rankings of the tested combinations according to best blood flow (e.g., vs. a target blood flow) and/or best blood flow and energy efficiency. The provided output of sets of stimulation parameter values and resultant blood flow indications may be further ranked using a patient pain score indicating the degree of pain relief the patient experiences with each combination of stimulation parameters. The blood flow information may be displayed on the programmer as an actual number, alpha or numeric rating. In some examples, the blood flow information may be displayed as high, medium, or low. In some examples, the blood flow information may be displayed as red, green, or yellow, e.g., with green indicating blood flow within a target range or close to a target blood flow value, yellow indicating blood flow closer to the limits of a target range or less close to a target blood flow value, and red indicating a blood flow that is outside of a target range or not close to a target blood flow value. Programmer 300 may present recommended parameter selections, such as parameter candidate sets, and expected blood flow for each candidate set and, in some examples, additional information such as energy and pain ratings as described above.

User-directed or automated selection of stimulation parameters, or recommended selection of stimulation parameters for the programmer may be based on the best blood flow achieved, or a combination of blood flow (vs. a baseline blood flow) range and energy stimulation parameters (e.g., stimulation parameters that reduce energy consumption by providing less amplitude, pulse width and/or frequency while achieving adequate blood flow). Selection based on blood flow and energy stimulation parameters could support the selection of low energy waveforms that provide enough energy/intensity to produce the desired blood flow, while avoiding excessive current consumption. In some cases, the selected stimulation parameters could be sub-threshold in that the stimulation parameters are below a level at which the patient perceives the stimulation (e.g., below a sensory perception threshold at which the patient can feel the stim in the form of paresthesia (e.g., tingling, numbness, or pressure) or other physical sensations), yet at a level sufficient to promote a desired level of blood flow. User-directed or automated selection of stimulation parameters with the programmer can be based in part on patient stimulation parameters, for example a pain rating input by a user for each tested parameter setting.

The architecture of external programmer 300 illustrated in FIG. 3 is shown as an example. The techniques as set forth in this disclosure may be implemented in the example external programmer 300 of FIG. 3, as well as other types of systems not described specifically herein. Nothing in this disclosure should be construed so as to limit the techniques of this disclosure to the example architecture illustrated by FIG. 3.

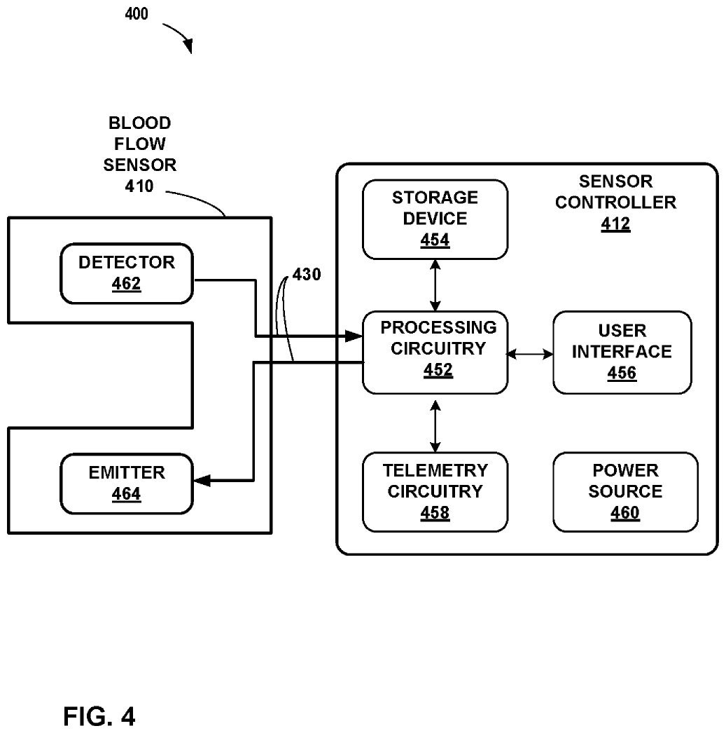

FIG. 4 is a block diagram illustrating an example of a blood flow sensing system 400 suitable for use with the IMD of FIGS. 2A and 2B and the programmer of FIG. 3, in accordance with one or more techniques of this disclosure. The blood flow sensing system 400 allows for a localized determination of blood flow in tissue, for example using non-invasive, laser, doppler, optical, or fluorescence techniques. In one or more examples, the blood flow sensing system 400 may include a laser speckle imaging systems, which may determine rate of movement of light scattering particles within a sample. In some examples, examples of blood flow sensing system 400 are shown in US 2019/0086316 and US 2020/0158548, which are incorporated herein by reference. In some examples, the sensor 410 monitors tissue that is distal to, i.e., remote from, a stimulation site. As an example, stimulation may be delivered to a first tissue site of the spinal cord as spinal cord stimulation, and blood flow may be sensed by blood flow sensor 410 at a second tissue site of a limb or appendage, where the first tissue site relates to first tissue, the second tissue site relates to second tissue and the first tissue is different than the second tissue. The blood flow sensing system 400 may include a blood flow sensing device such as sensor 410 including at least one of an external blood flow sensor or an implantable flood flow sensor. The sensor 410 is typically coupled with a patient or is disposed near the patient in order to allow the sensor to sense the blood flow. Examples of types of blood flow sensors include clip-on, slip-on, contact, adhesive, elastic banded, or implanted sensors. Sensor 410 may be located remote from the stimulation site, and disposed on or near one or more of a limb or an appendage, such as a leg, foot, toe, arm, hand, finger, wrist, earlobe, or nostril. As particular examples, sensor 410 may be located on a patient to detect blood flow at an upper extremity, such as a finger, or a lower extremity, such as a toe. In one or more examples, the blood flow sensing device may be housed separately from electrical stimulation circuitry, such as the IMD, and separately from the processing circuitry of the IMD.

The blood flow sensing system 400 includes a sensor 410, and a sensor controller 412. Sensor 410 can include a single sensor or multiple sensors, and may include an external sensor or an implantable sensor. Sensor 410 detects the blood flow data and communicates the blood flow data to the sensor controller 412. The sensor controller includes processing circuitry that receives the blood flow data from the sensor 410 and determines the blood flow values from the blood flow data. In some examples, the sensor 410 may be coupled to the sensor controller 412 via coupling 430, where the coupling may be a physical coupling such as cables, a combined cable. In some examples, coupling 430 is a wireless coupling, where the sensor 410 wirelessly communicates information to the sensor controller 412. In one or more examples, sensor 410 and sensor controller 412 may be integrated into a single housing.

The sensor controller 412 may include processing circuitry 452, storage device 454, user interface 456, telemetry circuitry 458, and power source 460. Storage device 454 may store instructions that, when executed by processing circuitry 452, cause processing circuitry 452 and sensor controller 412 to provide the functionality ascribed to the blood flow sensor throughout this disclosure. Each of these components, circuitry, or modules, may include electrical circuitry that is configured to perform some, or all of the functionality described herein. For example, processing circuitry 452 may include processing circuitry configured to perform the processes discussed with respect to processing circuitry 452. The sensor controller 412, or portions thereof, may reside in the programmer (FIG. 3), the IMD (FIG. 2A) or in an independent housing separate from the blood flow sensor 410.

In some examples, blood flow sensor 410 is configured to connect with tissue of a patient, such as an appendage, for example a finger or toe via an enclosure. The enclosure will fix the sensor to limit movement relative to the patient. In some examples, the enclosure includes a clip, strap, buckle, elastic band, tape, adhesives, wrap, or ties. In one or more examples, the enclosure may allow tissue to be slide therein, including a cylinder, box, rectangle, or sphere and blood flow is sensed while the tissue is disposed within the enclosure. In an example, the enclosure may be a spring loaded clip that claims on a finger or toe.

In some examples, blood flow sensing system 400 may include a blood flow sensor 410 that includes a source such as emitter 464 configured to emit light, and a detector 462 configured to detect at least a transmitted or reflected portion of the light emitted by the emitter 464 positioned within the blood flow sensor 410. Tissue such as an appendage from a patient is disposed between the detector 462 and the emitter 464. The emitter 464 may be chosen to maximize transmission of the light through the tissue. The detector 462 may include a camera, charge-coupled device (CCD) camera, complementary metal-oxide semiconductor (CMOS) camera, or a photodiode.

In use, in some examples, processing circuitry 452 directs emitter 464 to transmit light to transilluminate an appendage, e.g., transilluminate an entire thickness of the appendage. Detector 462 is configured to receive transmitted light after the light travels through the thickness of the appendage. Data from the detector 462 is transferred to processing circuitry 452.

Blood flow sensing system 400 further includes sensor circuitry configured to generate information relating to blood flow based on the detected portion of the light. Blood flow sensing system 400 may further include communication circuitry configured to transmit the blood flow information to processing circuitry 452 or the programmer, which processes the data and determines blood flow of the appendage.

The sensor controller 412 also, in various examples, may include a storage device 454, such as RAM, ROM, PROM, EPROM, EEPROM, flash memory, a hard disk, a CD-ROM, including executable instructions for causing the one or more processors to perform the actions attributed to them. Moreover, although processing circuitry 452 and telemetry circuitry 458 are described as separate modules, in some examples, processing circuitry 452 and telemetry circuitry 458 are functionally integrated. In some examples, processing circuitry 452, telemetry circuitry 458 or other circuitry of flow sensing system 400 correspond to individual hardware units, such as ASICs, DSPs, FPGAs, or other hardware units. Storage device 454 (e.g., a storage device) may store instructions that, when executed by processing circuitry 452, cause processing circuitry 452 and sensor controller 412 to provide the functionality ascribed to sensor controller 412 throughout this disclosure. In addition, storage device 454 may include a plurality of programs, where each program includes a parameter set that defines sensing timing or sensing coincidence.

In one or more examples, the system 400 may be programmed to sense substantially continuously over time. For example, the system 400 may be programmed to sense blood flow after electrical stimulation, every n minutes, with meals, after glucose dosing, while patient is sleeping, awake, standing, active or responsive to other events. In an example, the system 400 may be programmed to sense persistently over time, or intermittently over time, for instance at regular or irregular intervals. In one or more examples, for example in the case of persistently/continuously sensing blood flow, the sensor 410 could accompany the patient through their daily activities, e.g., as an implant or a wearable. In an example of when blood flow is sensed intermittently, a patient may attach the sensor at a particular time to get the blood flow information (e.g., once a week, once a day, every x hours, coincident with a glucose measurement or insulin dosage or meal), etc. In one or more examples, the patient may receive reminders from the system to attach the blood flow sensor 410 to obtain a reading. In one or more examples, the patient may receive reminders to log pain, diet, or activity along with a blood flow sensor reading.

Storage device 454 may also store data received from the sensor 410. In addition, storage device 454 may store data for a correlation index for the blood flow value in accordance various techniques described herein. In an example, system 400 may be programmed to store blood flow information and send the blood flow information wirelessly to a programmer or IMD using a variety of intervals or events. In some examples, system 400 may send blood flow information every n minutes, intermittently over time, or consistently over time. In some examples, system 400 may send blood flow information if the blood flow value falls below a certain threshold, if the blood flow value is out of a predetermined range of values, or if a rate of change in blood flow value exceeds a threshold.

Telemetry circuitry 458 may support wireless communication between the sensor 410 and sensor controller 412 under the control of processing circuitry 452. The sensor controller 412 may be configured to be under the control of the programmer 300 (FIG. 3) and/or be part of the programmer 300. Telemetry circuitry 458 may also be configured to communicate with another computing device via wireless communication techniques, or direct communication through a wired connection. In some examples, telemetry circuitry 458 provides wireless communication via an RF or proximal inductive medium. In some examples, telemetry circuitry 458 includes an antenna, which may take on a variety of forms, such as an internal or external antenna.

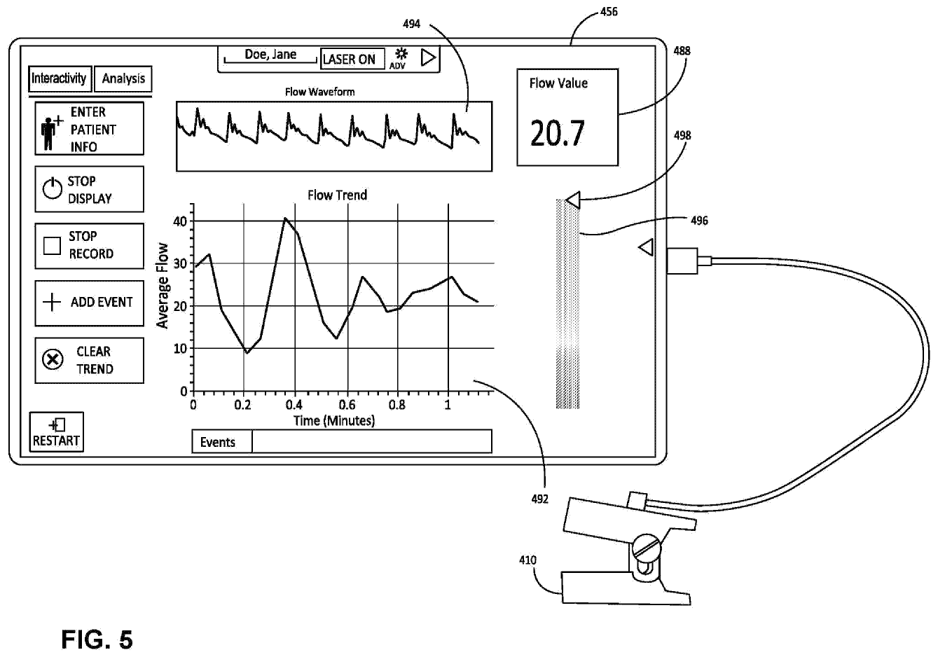

FIG. 5 is a conceptual diagram of the blood flow sensing device of FIG. 4. In an example, the sensor 410 is clipped on to an appendage of a patient such as a toe of a patient and sends blood flow information to the sensor controller 412. The sensor 410 may be disposed on a location on a patient at a location that is different or remote from the stimulation site. The sensor 410 may include multiple sensors which are disposed at unilateral or bilateral locations. In some examples, sensor 410 is housed in a clam shell that may include a spring mechanism and hinge, so that the sensor 410 can be releasably clipped or clamped onto the appendage. In some examples, sensor 410 may include slip-on, contact, adhesive, elastic, non-elastic, band, or implanted sensors.

User interface 456 allows for a user to input patient/subject information, and allows for a user to start and stop the output of blood flow information, as well as change view settings. User interface 456 may be in addition to the programmer user interface and in a separate device such that the clinician would use two devices, or the functionality of user interface 456 may be incorporated with the programmer interface so that the clinician would use a single device. In an example, when a user selects to start display, real time blood flow can be displayed. In some examples, blood flow data may be displayed as a function of time, which shows the flow waveform 494 and displays blood flow over a period of time, such as 10 s. The flow waveform may be updated in real time. In some examples, average flow may be computed every n seconds and plotted as flow trend 492. In addition, a blood flow value 488 may be displayed. In one or more examples, the blood flow value is in arbitrary units which may be linearly corrected with a volumetric flow. In another example, a blood flow indicator bar 496 is provided which indicates blood flow with a color map. The bottom-most portion of the indicator bar 496, which may be deep red in color, may indicate a minimum measurable flow. The top-most portion of the indicator bar 496, which may be green in color, may indicate a maximum measurable blood flow. In an example, arrow 498 may indicate where the current averaged blood flow data being collected fits along the range of measurable values.

The user interface shown in FIG. 5 is only one example of a user interface that may represent information relating to blood flow of a patient receiving electrical stimulation and other examples are contemplated. As one example, a user interface that represents information relating to blood flow of a patient receiving electrical stimulation may include a bar graph including a plurality of bars, a magnitude of each respective bar representing blood flow when the electrical stimulation is delivered via a particular electrode combination.

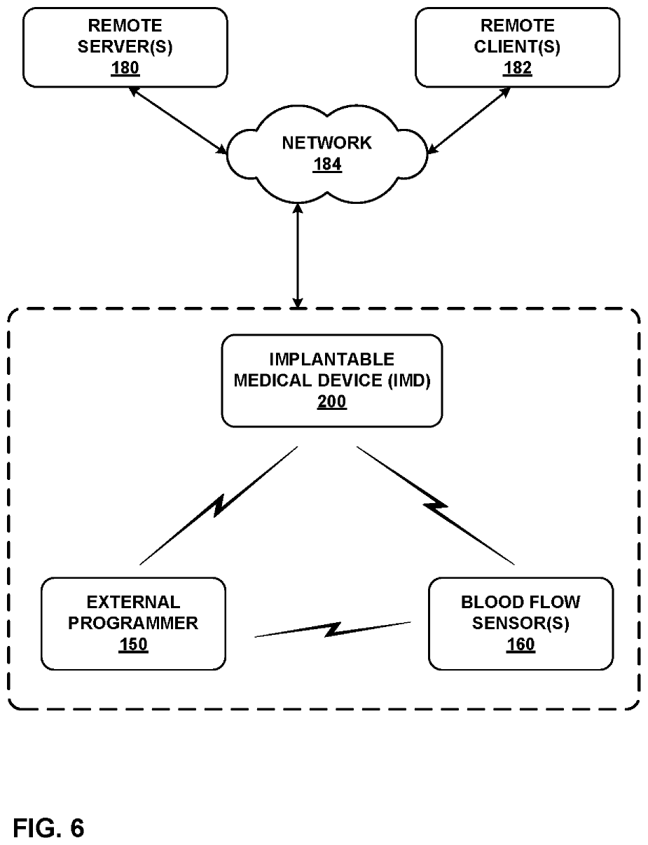

FIG. 6 is a block diagram of a system for evaluating efficacy of, assisting a user in programming, and/or automatically controlling neurostimulation or neurostimulation stimulation parameters using the techniques disclosed herein, including the use of blood flow information. In some examples, a remote system such as remote server 180 can receive parameter information and/or blood flow information via network 184 and may process the blood flow information, or may process the blood flow information in combination with parameter information. In some examples, remote server 180 may store the parameter information and/or blood flow information and process such information may be performed on a different remote server. The network 184 may comprise one or more wired and/or wireless networks. In some examples, network 184 may be the Internet.

In one or more examples, methods and use of the systems may be performed by a single device or among multiple devices located in separate locations. In an example, blood flow information from the blood flow sensor 160 and parameter information from the external programmer 150 or the implantable device is sent to the remote server 180 via the network 184. The remote server 180 may perform analysis over time on some or all of the received data to create correlation indices based on received data from a single patient or multiple patients. Remote server 180 processes the information to develop efficacy information, correlation indices, parameter recommendations, and communicates the processed information to the implantable stimulator or external programmer 150. In some examples, a clinician may view efficacy information, correlation indices, parameter recommendations via remote client 182 accessing remote server 180 and may program the IMD using the remote client 182 and the remote server 180. In some examples, the remote server 180 and may automatically program or control the IMD using the remote client 182 and the remote server 180 in closed loop control.

In one or more examples, a patient has a blood sensing device at home which checks blood flow persistently or intermittently over time, for instance at regular or irregular intervals, In one or more examples where a patient has a blood sensing device at home which checks blood flow persistently or intermittently, the blood sensing device may provide a notification of newly sensed blood flow information via network 184 to a remote client 182. Remote client 182 may prompt a clinician to check the newly sensed blood flow information and consider programming changes. The clinician may utilize a user interface of remote client 182 to review efficacy, enter stimulation parameter programming changes, and/or accept recommended stimulation parameter changes generated automatically by remote server 180. Remote server 180 can send the programming changes to the IMD 200 via network 184. In some examples, the remote server 180 may remotely retrieve blood flow information from the IMD, programmer, or blood flow sensing device, and may send programming directly to the IMD or to the IMD via the programmer. In some examples, the programmer, IMD, and/or blood flow sensing device communicate with the remote server 180 over a network connection through a network access device.

Although shown as separate entities, in some examples, functionality may be distributed differently than that shown in FIG. 6. For example, remote server 180 and remote client 182 may be the same system.

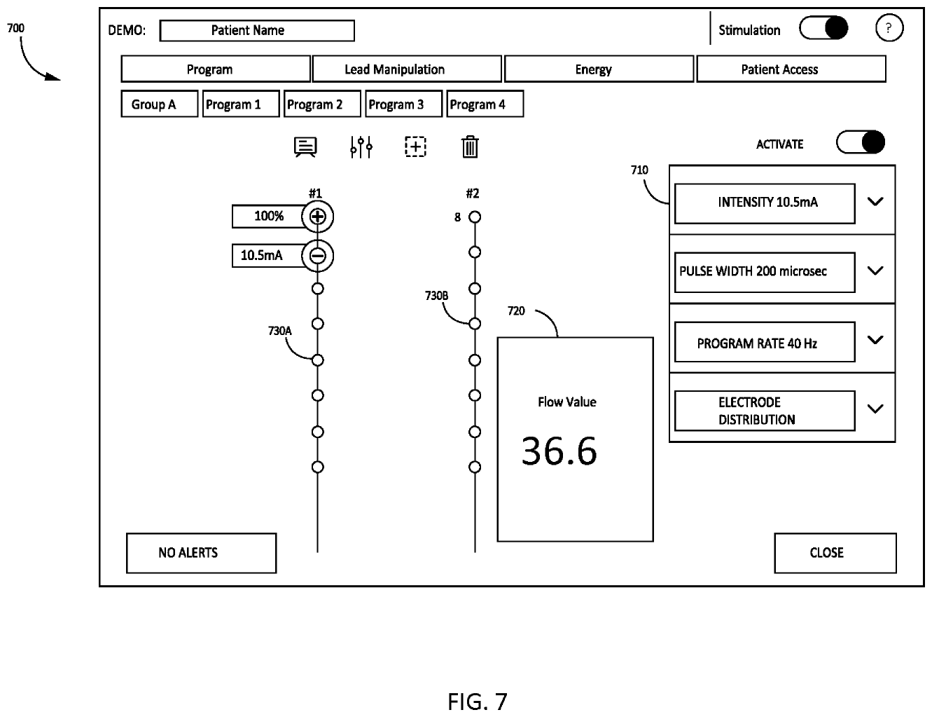

FIG. 7 is a diagram of a user interface 700 for an external programmer or remote monitoring/programming device suitable for use in the system of FIG. 6 and/or the programmer of FIG. 3. The user interface 700 allows for a user to receive information and to input information for the external programmer, and may form part of the programmer or a remote device for interacting with the controller. In one or more examples, the user interface 700 includes a display device.

In some examples, user interface 700 includes a display, for example comprising an LCD or LED display, and an input such as a keyboard, keypad, or touch screen. The user interacts with the programmer via the input of user interface 700. The user may also interact using peripheral pointing devices, such as a stylus scroll wheel, mouse, or any combination of such devices. The input includes parameter adjustment inputs 2 and/or electrode selection inputs 730A, 730B. Electrode selection inputs 730A, 730B allows the clinician to input which particular electrode to be used and further to specify the polarity of the electrodes. In one or more example, a user may use a single click for one polarity and double click for the other polarity. The input may further include patient information such as a pain rating, sensory rating.

User interface 700 further includes a blood flow output display 720 which may include a flow value and may allow for the user to monitor blood flow information as it corresponds to stimulation parameters. In some examples, the blood flow output display 720 shows raw blood flow data, change in blood flow data from a baseline blood flow, or raw blood flow data as a function of time, which shows the flow waveform and displays blood flow over a period of time, such as 10 s. The flow waveform may be updated in real time. In some examples, average flow is computed every n seconds and plotted and displayed as flow trend. In another example, blood flow output display 720 shows a blood flow indicator bar is provided which indicates blood flow with a color map showing a range of blood flow values (high and low), and a further shows an indicator where within the map the current blood flow value resides.

The parameter adjustment inputs 710 allow for a user to modify the stimulation parameters and visualize what stimulation parameters are currently implemented. Stimulation parameters include a selection of one or more electrodes, a polarity of each selected electrode, a voltage or current amplitude, a pulse width, and a pulse frequency as stimulation parameters.

User interface 700 communicates with the programmer and directs the IMD to test a number of parameter combinations while receiving blood flow information, and allows the user to identify particular parameter combinations that provide efficacious results. The user interface 700 communicates to programmer, for example, to direct the IMD to test a particular parameter at a particular value or range of values using the parameter adjustment inputs 710. In some examples, the user may manually select stimulation parameters to test and receive blood flow value for each test. In one or more examples, the user can select a program for automatically identifying parameter, combination of stimulation parameters, and stimulation parameter to test and receive blood flow value for each test. The user may use the blood flow information to determine if effective stimulation parameters have been selected for the patient, i.e., if the selected stimulation parameters produce stimulation that support therapeutic efficacy, e.g., in promoting desired blood flow, alleviating or reducing symptoms of a disease or disorder, or delaying the onset of symptoms or tissue damage or degeneration due to the disease or disorder. User interface 700 directs the programmer to control the IMD test stimulation with a given set of stimulation parameters, for example multiple sets of stimulation parameters and the resulting blood flow value for each of the multiple sets of stimulation parameters is displayed on the user interface 700. In one or more examples, user may input a target blood flow value and the user interface 700 displays a recommended set of stimulation parameters to test.

In an example, user inputs an intensity value at a first test value into user interface 700. The programmer receives the user input and directs stimulation to the IMD using the intensity level at the first test value, and user interface 700 displays the resulting blood flow value sensed during stimulation at the first test level. As additional changes to the intensity value are input into user interface 700, the user interface 700 displays blood flow values in the blood flow output display 720 achieved for each intensity level input by the user. Similarly, as positional changes to the stimulation value are input into user interface 700 by selecting different electrode combinations, the user interface 700 displays blood flow values in the blood flow output display 720 achieved for electrode combination input by the user. This technique may be used for various stimulation parameters such as electrode combination, a polarity of each selected electrode, a voltage or current amplitude, a pulse width, and a pulse frequency.

In some examples, a user may enter values for a combination of stimulation parameters. In an example, user inputs an intensity value and pulse width value at a first test values into user interface 700. The programmer receives the user input and directs stimulation to the IMD using the intensity level at the first test values, and user interface 700 displays the resulting blood flow value sensed during stimulation at the first test level. As additional changes to the intensity value and pulse width value are input into user interface 700, the user interface 700 displays blood flow values achieved for each intensity and pulse width value input by the user.

In another example, user interface 700 allows for a user to receive information and to input information for the external programmer that directs the IMD to manually or automatically test a sequence of electrode combinations or to select a program for automatically identifying the sequence of electrode combinations to test. The programmer controls the IMD to shift between each of the electrode combinations by shifting stimulation energy from a first electrode combination to a second electrode combination in incremental steps the user interface 700 displays the selected electrodes, electrode polarity, and blood flow information for each of the electrode combinations.

The shifting technique may be responsive to input from the user through the user interface 700. For example, programmer may require that the user input instructions via the user interface 700 to shift the incremental steps (up/down). The shifts may include shifting of electrode positions, for example among different electrode combinations, amplitude shifts for a given electrode combination, shifting or pulse width or rate for a given electrode combination, or a combination of these. If the user does not enter input into the user interface 700, programmer may not further adjust the pairings of the electrodes.

The subsequent electrode combination is the next electrode combination of the pre-defined or calculated electrode combination sequence. The subsequent electrode combination of the sequence may be an adjacent electrode combination. Adjacent electrode combinations include electrode combinations generated by shifting an electrode combination pattern upward or downward on a lead or by shifting left or right across columns in an array of leads or electrodes. For example, in a single lead numbered 0-7, the bipoles at 0-1 and 2-3 would be adjacent to the bipole at 1-2. For an array of electrodes or the parallel implant of linear leads, the bipole at 1-2 in a first column (or first linear lead) would be considered adjacent to the bipole in the second column at level 1-2.

The subsequent electrode combinations of the sequence, however, need not be adjacent electrode combinations. Although shifting between adjacent electrodes is the most likely use of this shifting feature, this feature could also be used to shift stimulation gradually between non-adjacent or unrelated combinations. This may be desirable in the case where the ‘adjacency’ in sensation does not directly correlate with adjacency on the lead, which may be due to nerve branching or other anatomical structure. Nonadjacent shifting may simply prove more pleasing to the patient than the traditional method of stopping one group of settings prior to beginning stimulation on a second group.

The user interface 700 is used as part of the external programmer or remote monitoring/programming device to allow the user to receive blood flow information, blood flow value for each shift of electrode combination and/or other parameter settings, which can support evaluation of efficacy and selection of stimulation parameters for programing the IMD. The provided output of resultant blood flow indications for each shift of electrode combination and/or other parameters may include raw blood flow values, relative scores (1-x) for blood flow, or rankings of the tested combinations according to best blood flow (e.g., vs. a target blood flow) and/or best blood flow and energy efficiency.

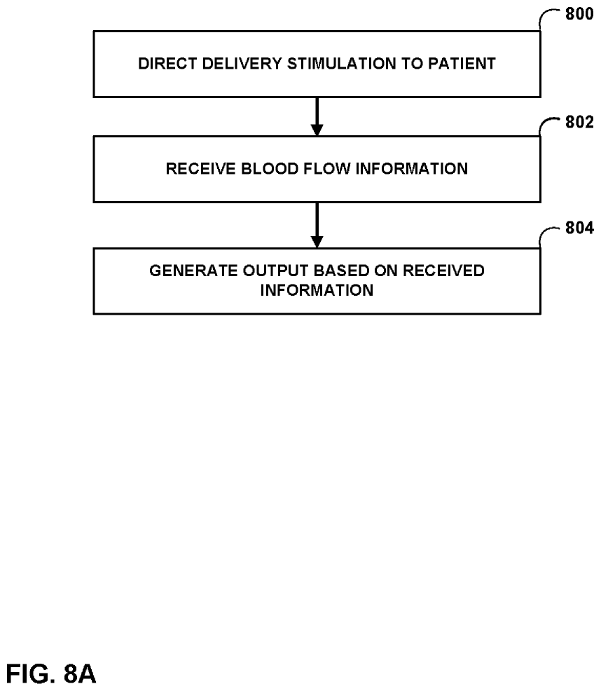

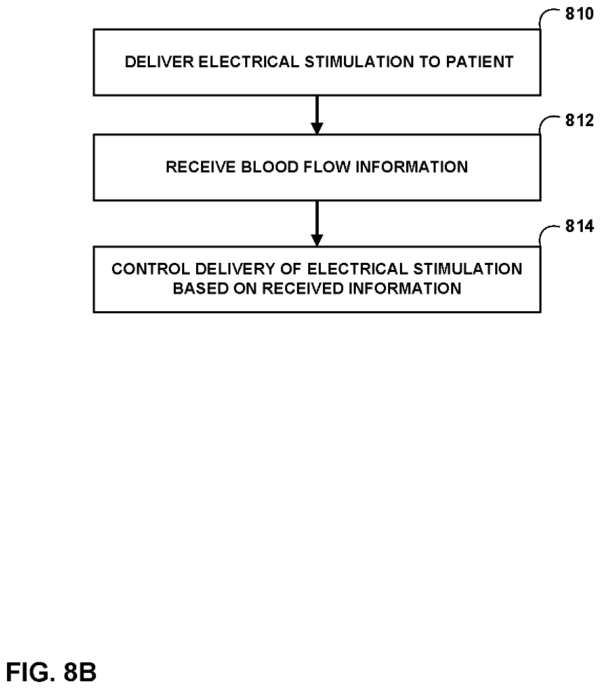

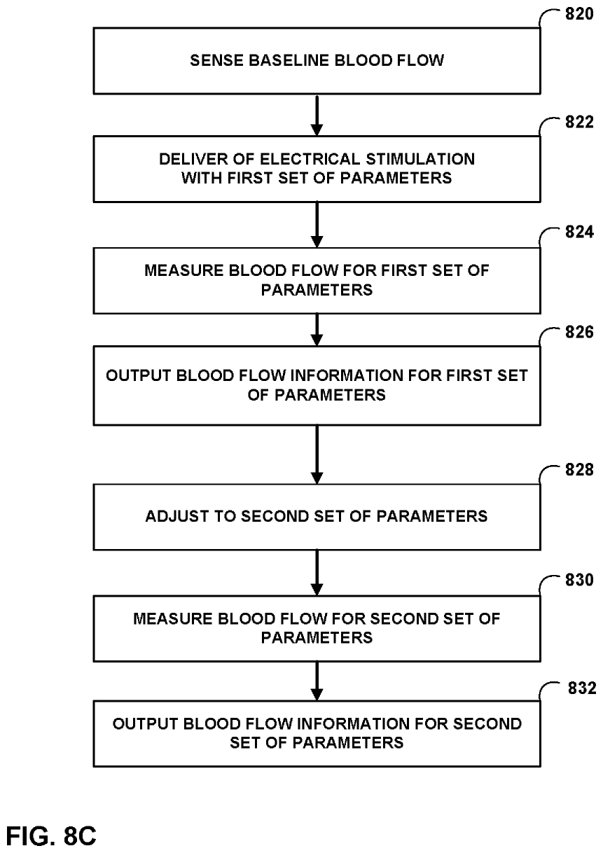

FIG. 8A is a flow diagram illustrating delivering electrical stimulation based on blood flow information. In an example, one or more processors may be configured to direct electrical stimulation to a patient (800), for example via electrodes to deliver the electrical stimulation generated by electrical stimulation circuitry. In one or more examples, the one or more processors directly control electrical stimulation, or indirectly control electrical stimulation by generating an instruction for indirect control of the electrical stimulation.

At 802, the processors may receive information relating to blood flow associated with tissue of the patient upon the delivery of the electrical stimulation to the patient, such as blood flow values. In one or more examples, the information may be collected with a blood flow sensing device configured to sense the blood flow associated with the tissue of the patient. The blood flow sensing device may include an external blood flow sensor or an implantable blood flow sensor. The received information may be sent and/or received from the blood flow sensing device via wireless telemetry.

The processors may generate output based on the received information (804). In one or more examples, the output may include blood flow values, and/or one or more electrical stimulation efficacy indications for the delivered electrical stimulation. In one or more examples, the output may include one or more recommended electrical stimulation parameters for the delivery of the electrical stimulation, such as one or more of electrode combination, stimulation amplitude, stimulation pulse width, stimulation frequency, or duty cycle. In one or more examples, the one or more processors may be configured to generate additional output including one or more of patient-indicated symptom relief, patient glucose level, patient activity level, patient posture, or stimulation energy efficiency. In one or more examples, the one or more processors may be configured to generate the output based on the received information via a user interface.

In one or more examples, the one or more processors may be configured to receive user input selecting one or more stimulation parameters or multiple sets of stimulation parameters of the electrical stimulation and direct delivery of the electrical stimulation based on the selected stimulation parameters or multiple sets of stimulation parameters, and optionally the output includes respective blood flow values for each of the multiple sets of stimulation parameters, and/or electrical stimulation efficacy indications for the delivered electrical stimulation based on the respective blood flow values for each of the multiple sets of stimulation parameters. In one or more examples, the one or more processors may be configured to store indications of the received information in association with the multiple sets of stimulation parameters. In one or more examples, the electrical stimulation includes one or more parameters selected to deliver therapy to address a condition of one or more of painful diabetic neuropathy (PDN), peripheral vascular disease (PVD), peripheral artery disease (PAD), complex regional pain syndrome (CRPS), angina pectoris (AP), leg pain, back pain or pelvic pain. The output may be used to develop recommended stimulation parameters, and be presented to a user (such as a clinician or patient) and/or automatically implemented.

FIG. 8B is a flow diagram illustrating controlling electrical stimulation based on blood flow information. In an example, electrical stimulation circuitry may be configured to generate electrical stimulation to a patient, for example via electrodes to deliver the electrical stimulation generated by electrical stimulation circuitry (810). In one or more examples, one or more processors directly control electrical stimulation, or indirectly control electrical stimulation by generating an instruction for indirect control of the electrical stimulation. At 812, the processing circuitry, which may include processors, may receive information relating to blood flow associated with tissue of the patient upon the delivery of the electrical stimulation to the patient, such as blood flow values. In one or more examples, the blood flow information may be collected with a blood flow sensing device configured to sense the blood flow associated with the tissue of the patient. The blood flow sensing device may include an external blood flow sensor or an implantable blood flow sensor. The received information may be sent and/or received from the blood flow sensing device via wireless telemetry.

The processing circuitry may automatically control the electrical stimulation circuitry to deliver the electrical stimulation to the patient based on the received information (814). In one or more examples, the received information may include blood flow values. The received information may further include patient information, such as by patient pain score, stimulation perception score, patient activity level, posture, glucose level, body temperature, time of day, diet, and other input from patient sensors, such as accelerometers. In one or more examples, the processing circuitry is configured to control the electrical stimulation circuitry to deliver the electrical stimulation to the patient based on multiple instances of received blood flow information over time. In one or more examples, the processing circuitry may be configured to adjust one or more stimulation parameters of the electrical stimulation based on the received information, and control the electrical stimulation circuitry to deliver the electrical stimulation based on the adjusted stimulation parameters. In an example, the processing circuitry may adjust one or more stimulation parameters of the electrical stimulation based on the received information, and control the electrical stimulation circuitry to deliver the electrical stimulation based on the adjusted stimulation parameters.

In one or more examples, the processing circuitry may be configured to adjust one or more of the stimulation parameters of the electrical stimulation if the received information indicates blood flow value is outside a range of blood flow values, is below a minimum blood flow value, or exceeds a maximum blood flow value. In one or more examples, the processing circuitry may be configured to adjust one or more of the stimulation parameters of the electrical stimulation to achieve a desired blood flow over a period of time, for example for a post-operative patient to reduce habituation or desensitization to the stimulation. In one or more examples, the processing circuitry may be configured to adjust a duty cycle of the stimulation parameters of the electrical stimulation to achieve the desired blood flow over a period of time to reduce desensitization to the stimulation. In one or more examples, the processing circuitry may be configured to further adjust and/or deliver stimulation conditional on patient information such as patient posture and/or other information such as time of day. In one or more examples, the electrical stimulation includes one or more parameters selected to deliver therapy to address a condition of one or more of painful diabetic neuropathy (PDN), peripheral vascular disease (PVD), peripheral artery disease (PAD), complex regional pain syndrome (CRPS), angina pectoris (AP). leg pain, back pain or pelvic pain.

FIG. 8C is a flow diagram illustrating determining efficacy of neurostimulation based on sensed blood flow information. The programmer will test one or more combinations of stimulation parameters, collecting and storing information relating to blood flow measured during stimulation at each of the parameter settings. The information relating to the measured blood flow at each of the stimulation parameters is used to determine neurostimulation efficacy. Blood flow may be automatically logged with each adjustment of parameters, the changes to blood flow may be identified by plotting blood flow over time.

A programmer, such as that shown in FIG. 3, collects blood flow information from a blood sensing device, where the blood sensing device senses a baseline blood flow (820). In an example, the baseline blood flow is a blood flow measurement while a patient is at rest and without stimulation. The programmer receives configuration information from a user, which may be used to deliver stimulation using an initial set of stimulation parameters, a first set of stimulation parameters (822). Stimulation parameters may relate to electrode combination, electrode polarity, amplitude, pulse width, pulse frequency, and duty cycle. Upon delivery of the electrical stimulation with the first set of stimulation parameters, blood flow is measured (824) for the first set of stimulation parameters. At 826, information for the measured blood flow is output, for example to the programmer, and stored. The output for each measured blood flow may be displayed to a user for each set of stimulation parameters.

At 828, the programmer is used to adjust the IMD to deliver stimulation at a second set of stimulation parameters that are different than the first set of stimulation parameters. For example, the second set of stimulation parameters may have a different amplitude setting than the first set of stimulation parameters, and the remaining stimulation parameters have the same value. In an example, the second set of stimulation parameters may have a different electrode combination than the first set of stimulation parameters. The user may input adjustments to the programmer to adjust from the first set of stimulation parameters to the second set of stimulation parameters. Programmer implements the second set of stimulation parameters, and directs electrical stimulation to be delivered to the patient using the second set of stimulation parameters. Upon delivery of the stimulation, blood flow is measured for the second set of stimulation parameters (830). The measured blood flow information for stimulation delivered at the second set of stimulation parameters is output to the programmer (832). This process may be repeated with additional combinations of stimulation parameters, for example for a third set, fourth set, etc.

In determining efficacy of the neurostimulation, the blood flow information is correlated to the parameter settings, and evaluated relative to the parameter settings, where a degree of increased blood flow typically indicates efficacy of the parameter setting. In some examples, the parameter settings may be ranked by blood flow readings. By ranking the blood flow readings, a list of stimulation parameters that produce a desired blood flow may be developed, identifying particularly efficacious parameter settings. For example, ranking of the blood flow readings from high to low may determine efficacy of lead placement by ranking the various readings obtained for various electrode positions. In one or more examples, ranking of the blood flow readings from high to low and including ranges of acceptable blood flow readings will determine efficacy of multiple parameter combinations such as, but not limited to stimulation amplitude, pulse width and pulse frequency. In one or more examples, blood flow readings may be further ranked by patient information, such as by patient pain score, stimulation perception score, patient activity level, posture, glucose level, body temperature, time of day, diet, and other input from patient sensors, such as accelerometers. The programmer may provide efficacy ratings for the various parameters to be viewed by a clinician, or the user can view blood flow to evaluate the efficacy of the different settings.

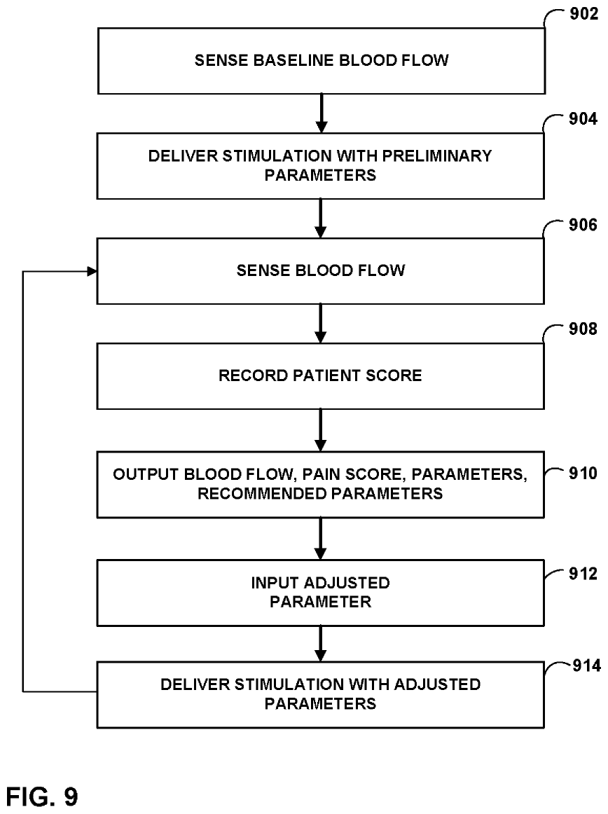

FIG. 9 is a flow diagram illustrating programming of one or more neuro stimulation stimulation parameters based on sensed blood flow information. The programmer may be used to determine efficacy of particular parameter settings of the IMD by testing parameter settings, for example for multiple sets of stimulation parameters, such as a first set, second set, and third set of parameters, and recording blood flow for each parameter setting, and implementing the parameter settings. The user could manually advance scanning through electrode pairs or parameter combinations. As the user scans through the electrode pairs or parameter combinations to test and record blood flow values for each combination, the user may optionally collect a patient pain score indicating the degree of pain relief information from the combination, or a stimulation perception score indicating whether the patient perceives the stimulation.

In one or more examples, programmer directs the blood flow sensor to sense and store a base line blood flow for a patient without electrical stimulation (902). The base line blood flow is sensed from tissue prior to delivering stimulation. After the base line blood flow is collected, stimulation is delivered to the patient at a set of stimulation parameters (904), where the stimulation parameters include electrode combination, a polarity of each selected electrode, a voltage or current amplitude, a pulse width, and a pulse frequency. Upon delivery of the stimulation at the stimulation parameter, blood flow is sensed for the stimulation parameter and blood flow information is collected (906). Patient information is collected while the patient is being stimulated using the current parameter setting, where the patient information may include subjective information such as level of pain perception or perception of stimulation, for example whether the patient feels the stimulation. The blood flow information and patient information are stored with the stimulation parameters, and the blood flow information and patient information are output to the user.

User manually inputs changes to stimulation parameters, and directs the programmer to test the adjusted stimulation parameters (912, 914). For example, the programmer directs the IMD to deliver stimulation with an adjusted amplitude, for example with an amplitude different than the preliminary amplitude values, and the blood flow is sensed. The user collects patient information for the adjusted stimulation parameters, such as patient pain or perception information. The parameter information, patient information and corresponding blood flow information is output to the user and also stored. The user may continue to shift the amplitude values by either increasing or decreasing the amplitude and observing the respective blood flow values and patient feedback. While the example of amplitude is provided, the user may direct stimulation circuitry to step through various incremental settings of other stimulation parameters, such as stimulation pulse width, stimulation frequency, or duty cycle, and record the respective blood flow information for each stepped value. The user may shift more than one stimulation parameter for each test and collect blood flow information for the multiple shifted stimulation parameters.

In some examples of inputting and testing stimulation parameters against blood flow, the programmer is used to determine efficacy of lead placement. A series of testing of electrode combinations may occur so that a user may identify desirable combinations based at least in part by the blood flow information. The programmer may provide a recommended electrode combination to the user. The user directs the IMD to scan though each of a plurality of electrode combinations, and for each combination, a sensed blood flow value is recorded for the particular electrode combination. The programmer or the IMD shifts stimulation energy between different electrode combinations, and monitors changes in blood flow as a result of the different electrode combinations. The user may also add for each electrode combination, a parameter combination and the sensed blood flow value is recorded for the particular electrode combination and parameter combination.

As an illustration for electrode pairing for the blood flow testing, a 2×8 electrode arrangement in which two leads each carry eight electrodes, and the electrodes on one lead are designated 0 through 7 from top to bottom, and the electrodes on the other lead are designated 8-15 from top to bottom, a first combination could be the following: 0+1−2+, where the number designates the electrode position and the plus or minus designates the polarity of the electrode.

In this example, a shift to a second electrode combination could yield the same pattern but simply move down one electrode position, e.g., 1+2−3+. In other embodiments, the first and second electrode combinations may have different patterns, e.g., combination 1=0+1− and combination 2=0+1−2+, and then combination 3=1+2−. Combinations between the leads (e.g., 1-7) could also be tested. For each combination of electrode pairing, different parameter settings could be tested, e.g., for electrodes 0-1, a set of different amplitudes, different pulse widths, or different frequencies, or combinations thereof. In one or more examples, unipolar combinations are paired and tested, where a single electrode 16 on the lead and an electrode on IMD housing (e.g., 0-16, 1-16, 2-16 . . . ). In one or more examples, multipolar combinations are tested, with three of more electrodes in various patterns (for example, 0-1-7, where 0 and 7 are cathodes and 1 is an anode).

For each of the multiple sets of stimulation parameters such as the electrode combinations and parameter combinations, the corresponding blood flow value is sensed and stored, where the sensed and stored blood value is recorded as being associated with the particular electrode combination and parameter combination. The user may interact with the programmer to indicate points at which the stimulation parameters of stimulation yield efficacious results, for example, with higher blood flow values. Programmer stores the stimulation parameters where the user has indicated efficacious results. For example, the programmer may store the stimulation parameters relating to electrode pairing, a polarity of each selected electrode, a voltage or current amplitude, a pulse width, and a pulse frequency for each set of stimulation parameters indicating of efficacious results in terms of resultant blood flow.

In one or more examples, the programmer presents to the user a list of the electrode combinations with associated blood flow indications, or a list of combined electrode and parameter combinations with associated blood flow indications so that the user can select one of them. Programmer may also highlight recommended combinations based upon predetermined priorities such as maximizing blood flow with best energy efficiency. The provided output of parameter combinations and blood flow indications may include raw blood flow values, relative scores (1-x) for blood flow, or rankings of the tested combinations according to best blood flow and/or best blood flow and energy efficiency. The provided output of parameter combinations and blood flow indications may be further ranked using a patient pain score indicating the degree of pain relief the patient experiences with each combination. The blood flow information may be displayed on the programmer as an actual number, alpha or numeric rating. In some examples, the blood flow information may be displayed as high, medium, or low. In some examples, the blood flow information may be displayed as red, green, or yellow. Programmer may present recommended parameter selections such as parameter candidates, and expected blood flow for each candidate.

The techniques may be performed in a clinic, or on a remote location where a patient has an ability to check blood flow with a blood flow sensing device. The blood flow sensing device may check blood flow persistently or periodically, and in some examples the blood flow information may be stored. In some examples, a clinician may be notified to check sensed blood flow information when new blood flow information is available, or if changes in blood flow information are detected, or if blood flow falls below a threshold. Recommended parameter changes based on the blood flow information may be presented to the clinician as part of the notification.

The clinician selects parameters or accepts recommended parameters based on the sensed blood flow. In some examples, the clinician selects parameters based on the sensed blood flow and patient score, such as a patient pain score, or energy efficiency, etc. The programmer programs the IMD to deliver the stimulation with the selected or accepted parameters.

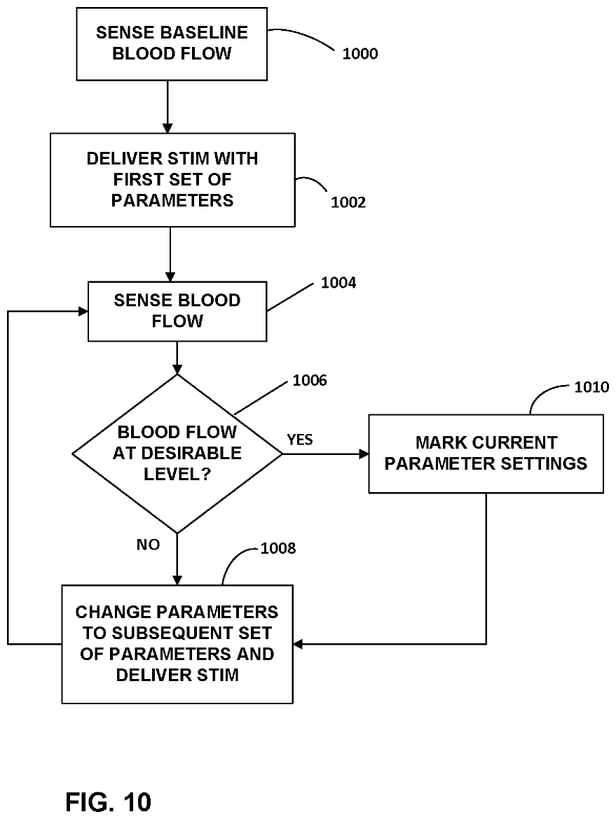

FIG. 10 is a flow diagram illustrating automated review of one or more neurostimulation stimulation parameters versus sensed blood flow information to support programming of neurostimulation stimulation parameters. A programmer may shift through different parameter settings automatically or semi-automatically rather than the user manually selecting each of them.

Programmer may be used to determine efficacy of particular parameter settings of the IMD by testing parameter settings and recording blood flow for each parameter setting, and giving a user an option to implement recommended parameter settings. The programmer automatically advances scanning through electrode pairs or parameter combinations to identify the electrode pairs or stimulation parameters that achieve a desired range of blood flow values, exceeds a minimum blood flow value, or falls below a maximum blood flow value.

In one or more examples, programmer directs the blood flow sensor to sense and store a base line blood flow for a patient without electrical stimulation (1000). The base line blood flow is sensed from tissue prior to delivering stimulation. After the base line blood flow is collected, stimulation is delivered to the patient at a set of stimulation parameters (1002), where the stimulation parameters include electrode combination, a polarity of each selected electrode, a voltage or current amplitude, a pulse width, and a pulse frequency. Upon delivery of the stimulation at the first set of stimulation parameters, blood flow is sensed for the stimulation parameter and blood flow information is collected (1004). The programmer evaluates whether the blood flow falls below a threshold blood flow value, increases beyond an upper limit for blood flow value, or falls outside of a range of values. In an example, the programmer evaluates a change in blood flow as compared to the sensed baseline blood flow. The blood flow information and patient information are stored with the stimulation parameters, and the blood flow information and patient information are output to the user.

If blood flow is not at a desirable level, for example, does not fall within a target range, is above a maximum level, or is below a minimum level, the programmer automatically selects a subsequent set of stimulation parameters and provides directions to the IMD to test the adjusted stimulation parameters by delivering stimulation with the adjusted stimulation parameters (1008). For example, the programmer directs the IMD to deliver stimulation with an adjusted amplitude, for example with an amplitude different than the preliminary amplitude values. Upon delivery of the stimulation with the adjusted stimulation parameters, the blood flow is sensed (1004). If blood flow is at a desirable level, the parameter selection is marked as an efficacious parameter set, and stored for review by a clinician and/or for use in automatically selecting parameter settings (1010).

The programmer continues to shift the stimulation parameters by either increasing or decreasing the stimulation parameter and collecting information regarding the respective blood flow values. In an example, amplitude values can be modified, keeping the remaining stimulation parameters constant. While the example of amplitude is provided, the programmer may direct stimulation circuitry to step through various incremental settings of other stimulation parameters, such as stimulation pulse width, stimulation frequency, or duty cycle, and record the respective blood flow information for each stepped value. The programmer may shift more than one stimulation parameter for each test and collect blood flow information for the multiple shifted stimulation parameters. The stepped testing may occur for a predetermined number of shifts in stimulation parameters. For example, the individual stimulation parameters may each be tested ten times, shifting a certain percentage each time.

In some examples of inputting and testing stimulation parameters against blood flow, the programmer is used to determine efficacy of lead placement. A series of testing of electrode combinations may occur so that the programmer may identify electrode combinations based at least in part by the blood flow information. In an example, the programmer may identify two or more electrode combinations achieving the greatest blood flow during stimulation, and two or more electrode combination achieving the least blood flow during stimulation to identify the best electrode combinations and the worst electrode combinations. The programmer directs the IMD to scan though each of a plurality of electrode combinations, and for each combination, a sensed blood flow value is recorded for the particular electrode combination. The programmer shifts stimulation energy between different electrode combinations, and monitors changes in blood flow as a result of the different electrode combinations. The programmer may also add for each electrode combination, a parameter combination and the sensed blood flow value is recorded for the particular electrode combination and parameter combination.

As an illustration for electrode pairing for the blood flow testing, a 2×8 electrode arrangement in which two leads each carry eight electrodes, and the electrodes on one lead are designated 0 through 7 from top to bottom, and the electrodes on the other lead are designated 8-15 from top to bottom, a first combination could be the following: 0+1−2+, where the number designates the electrode position and the plus or minus designates the polarity of the electrode.

In this example, a shift to a second electrode combination could yield the same pattern but simply move down one electrode position, e.g., 1+2−3+. In other embodiments, the first and second electrode combinations may have different patterns, e.g., combination 1=0+1− and combination 2=0+1−2+, and then combination 3=1+2−. Combinations between the leads (e.g., 1-7) could also be tested. For each combination of electrode pairing, different parameter settings could be tested, e.g., for electrodes 0-1, a set of different amplitudes, different pulse widths, or different frequencies, or combinations thereof. In one or more examples, unipolar combinations are paired and tested, where a single electrode 16 on the lead and an electrode on IMD housing (e.g., 0-16, 1-16, 2-16 . . . ). In one or more examples, multipolar combinations are tested, with three of more electrodes in various patterns (for example, 0-1-7, where 0 and 7 are cathodes and 1 is an anode).

For each of the electrode combinations and parameter combinations, the corresponding blood flow value is sensed and stored, where the sensed and stored blood value is recorded as being associated with the particular electrode combination and parameter combination. Programmer stores the stimulation parameters where the user has indicated efficacious results, and for stimulation parameters where a user has marked the stimulation parameters. For example, the programmer may store the stimulation parameters relating to electrode pairing, a polarity of each selected electrode, a voltage or current amplitude, a pulse width, and a pulse frequency for each indicating of efficacious results.

In one or more examples, after the automated scanning of the electrode pairings and stimulation parameters, the programmer presents to the user a list of the electrode combinations with associated blood flow indications, or a list of combined electrode and parameter combinations with associated blood flow indications so that the user can select one of them. Programmer may also highlight recommended combinations based upon predetermined priorities such as maximizing blood flow with best energy efficiency, e.g., in terms of stimulation amplitude, pulse width and/or pulse rate. The provided output of parameter combinations and blood flow indications may include raw blood flow values, relative scores (1-x) for blood flow, or rankings of the tested combinations according to best blood flow and/or best blood flow and energy efficiency. The blood flow information may be displayed on the programmer as an actual number, alpha or numeric rating. In some examples, the blood flow information may be displayed as high, medium, or low. In some examples, the blood flow information may be displayed as red, green, or yellow. Programmer may present recommended parameter selections such as parameter candidates, and expected blood flow for each candidate.

The techniques may be performed in a clinic, or on a remote location where a patient has an ability to check blood flow with a blood flow sensing device. The blood flow sensing device may check blood flow persistently or periodically, and in some examples the blood flow information may be stored. In some examples, a clinician may be notified to check sensed blood flow information when new blood flow information is available, or if changes in blood flow information are detected, or if blood flow falls below a threshold. Recommended parameter changes based on the blood flow information may be presented to the clinician as part of the notification.

The system allows for recording patient blood flow as it corresponds with stimulation parameters without interacting with the patient for patient input, allowing the clinician to obtain objective efficacy information of the stimulation parameters. This may be helpful particularly when a patient is asleep, such as during surgery, or otherwise not focused on the programming process, or when information is collected over a longer period of time, and avoids situations where a patient forgets to enter patient information since the information is automatically recorded.

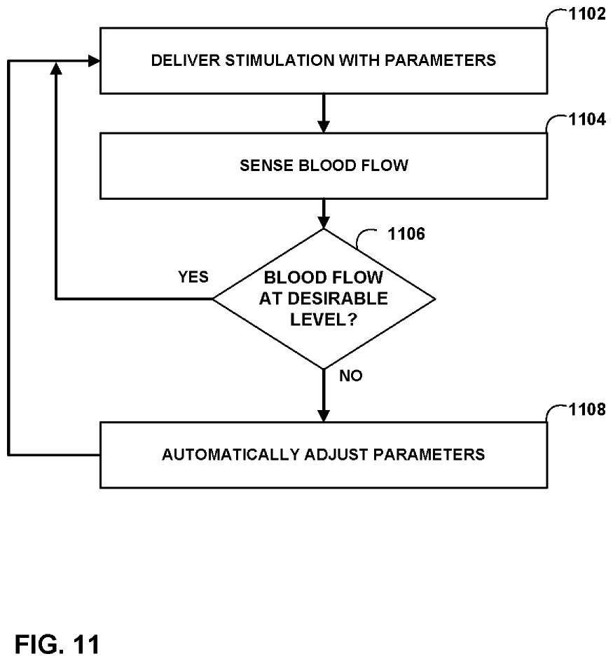

FIG. 11 is a flow diagram illustrating automated control of one or more neurostimulation stimulation parameters based on sensed blood flow information. The process may relate to a closed loop control where IMD or the external programmer receives and processes sensed blood flow information and automatically makes parameter adjustments.

Programmer may be used to determine efficacy of particular parameter settings of the IMD by testing parameter settings and recording blood flow for each parameter setting, and automatically implementing the parameter settings. The programmer automatically advances scanning through electrode pairs or parameter combinations to identify the electrode pairs or stimulation parameters that achieve a desired range of blood flow readings.

Stimulation is delivered to the patient at a set of stimulation parameters (1102), where the stimulation parameters include electrode combination, a polarity of each selected electrode, a voltage or current amplitude, a pulse width, and a pulse frequency. Upon delivery of the stimulation at the first set of stimulation parameters, blood flow is sensed for the stimulation parameter and blood flow information is collected (1104). In one or more examples, sensing blood flow occurs in real-time, such as a patient wears a blood flow sensing device full time, or has an implanted blood flow sensing device. In one or more examples, sensing blood flow occurs periodically, where blood flow is sensed n times per day or n times per week. The patient may periodically check blood flow, for example, at the instruction of an external device, based on an event, or coinciding with other medical events, and the parameter settings are automatically updated by the processing circuitry.

The techniques may be performed in a clinic, or on a remote location where a patient has an ability to check blood flow with a blood flow sensing device. The blood flow sensing device may check blood flow persistently or periodically, and in some examples the blood flow information may be stored. In some examples, a clinician may be notified to check sensed blood flow information when new blood flow information is available, or if changes in blood flow information are detected, or if blood flow falls below a threshold.

The programmer evaluates whether the blood flow is at a desirable level, for example falls below a threshold blood flow value, increases beyond an upper limit for blood flow value, or falls outside of a range of values. In an example, the programmer evaluates a change in blood flow as compared to a sensed baseline blood flow. The programmer may generate notifications based on changes in sensed blood flow in response to stimulation. If blood flow is at a desirable value, the parameter selection is maintained until a later blood flow measurement is not at a desirable value. If blood flow is not at a desirable level, the programmer automatically selects a subsequent set of stimulation parameters and provides directions to deliver stimulation with the adjusted stimulation parameters (1108). For example, the programmer directs the IMD to deliver stimulation with an adjusted amplitude, for example with an amplitude different than the preliminary amplitude values, or a different electrode combination. Upon delivery of the stimulation with the adjusted stimulation parameters, the blood flow is sensed (1104). In selecting a subsequent set of stimulation parameters, the programmer may choose from a list of stimulation parameters that have in previous testing resulted in achieving a blood flow within a target range, for example as provided in a correlation index.

If the sensed blood flow continues to test outside of the desirable level, the programmer continues to shift the stimulation parameters by either increasing or decreasing the stimulation parameter (and/or changing the electrode combination) and collecting information regarding the respective blood flow values. In an example, amplitude values can be modified, keeping the remaining stimulation parameters constant. While the example of amplitude is provided, the programmer may direct stimulation circuitry to step through various incremental settings of other stimulation parameters, such as stimulation pulse width, stimulation frequency, or duty cycle, and evaluating the respective blood flow information for each stepped value. The programmer may shift more than one stimulation parameter for each test and collect blood flow information for the multiple shifted stimulation parameters.

In some examples adjusting stimulation parameters, the programmer may change the electrode combination and/or polarity. If the sensed blood flow continues to be at an undesirable level, a series of testing of electrode combinations may occur so that the programmer may identify electrode combinations based at least in part by the blood flow information. In an example, when the programmer identifies an electrode combination that elicits a sensed blood flow that satisfies the desirable level, the electrode combination is automatically implemented.

In an example, an IMD delivers stimulation with stimulation parameters that include having a first electrode combination, and the sensed blood flow does not satisfy the desirable level. The programmer automatically directs the IMD to change from a first electrode combination to a second combination, and to deliver stimulation with the updated electrode combination. The sensed blood flow value is recorded for the particular electrode combination and parameter combination.

As an illustration for electrode pairing, a 2×8 electrode arrangement in which two leads each carry eight electrodes, and the electrodes on one lead are designated 0 through 7 from top to bottom, and the electrodes on the other lead are designated 8-15 from top to bottom, a first combination could be the following: 0+1−2+, where the number designates the electrode position and the plus or minus designates the polarity of the electrode.

In this example, a shift to a second electrode combination could yield the same pattern but simply move down one electrode position, e.g., 1+2−3+. In other embodiments, the first and second electrode combinations may have different patterns, e.g., combination 1=0+1− and combination 2=0+1−2+, and then combination 3=1+2−. Combinations between the leads (e.g., 1-7) could also be tested. For each combination of electrode pairing, different parameter settings could be tested, e.g., for electrodes 0-1, a set of different amplitudes, different pulse widths, or different frequencies, or combinations thereof. In one or more examples, unipolar combinations are paired and tested, where a single electrode 16 on the lead and an electrode on IMD housing (e.g., 0-16, 1-16, 2-16 . . . ). In one or more examples, multipolar combinations are tested, with three of more electrodes in various patterns (for example, 0-1-7, where 0 and 7 are cathodes and 1 is an anode).

In one or more examples, after the automated scanning stimulation parameters, which may include electrode pairings, the programmer may automatically implement parameter settings and/or electrode combinations which achieve a desired blood flow. The programmer may also automatically adjust stimulation parameters based upon predetermined priorities such as maximizing blood flow with best energy efficiency, e.g., in terms of stimulation amplitude, pulse width and/or pulse rate.

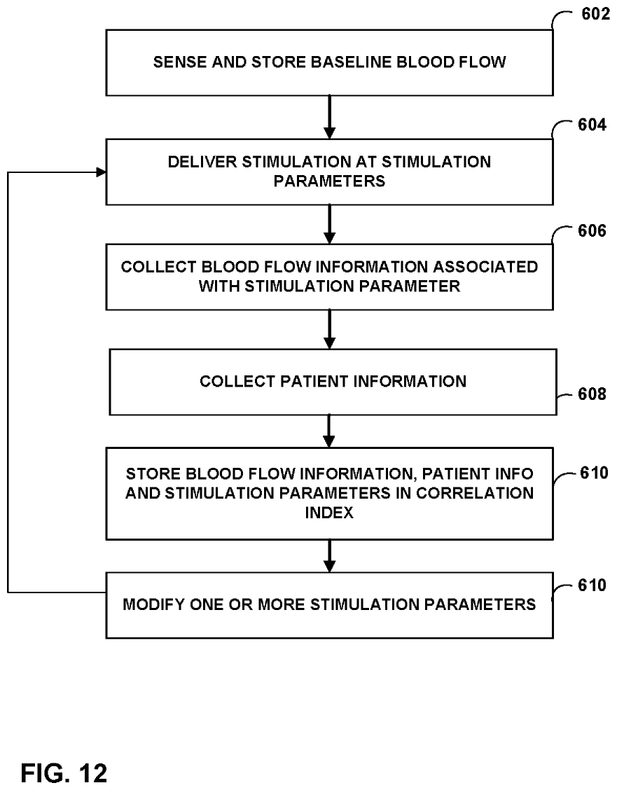

FIG. 12 is a flow diagram illustrating generation of index information based on correlation of one or more neurostimulation stimulation parameters and sensed blood flow information. Information relating to blood flow is stored over time as blood flow information is collected during stimulation. The blood flow information is stored by parameter settings and patient information occurring during a particular parameter setting to achieve the blood flow during stimulation. The information may be patient specific, or cover a population of patients with potentially related medical histories. The information may be used to develop a blood flow correlation index which may be used in a closed loop setting, where it is possible to automatically update and adjust patient stimulation parameters for the IMD. The clinician programmer or another device may generate the correlation index and may download the index to the IMD, or the clinician programmer or another device generates and stores the index and uses the index to direct or control the IMD. The blood flow correlation index may include a matrix of information that tracks a relationship between two or more variables. For example, the variables may include blood flow information for stimulation using a set of stimulation parameters, and the stimulation parameters may include electrode positions, combinations and polarities, or stimulation amplitude, pulse width, pulse rate, or cycling. In an example, raw blood flow data (mL/min) for each stimulation may be stored for each parameter setting. For example, a first blood flow data is stored for stimulation with a first set of stimulation parameters, and a second blood flow data is stored for stimulation for a second set of stimulation parameters. In addition, the blood flow values may be compared to a baseline blood flow value, and differences between the blood flow values under stimulation and the baseline blood flow may be stored in the blood flow correlation index.

The first and second blood flow values achieved using the first and second set of stimulation parameters may be further categorized within the blood flow correlation index by additional factors such as factors dependent on the patient, such as activity level, posture, glucose level, diet, pain input, input from patient sensors, such as accelerometers. Additional patient information can include factors such of day, body temperature, or increments of time. The correlation index may include a log of blood flow over time, and also after stimulation parameter settings have been adjusted.

Hence, the correlation index may include, as inputs, target blood flow values and, as outputs, corresponding sets of stimulation parameters expected to produce the target blood flow values. As additional inputs, the correlation index may include factors such as those discussed above, e.g., activity level, posture, glucose level, diet, pain input, input from patient sensors, day, body temperature, and/or increments of time. The outputs may be used to develop recommended parameters or parameters that are automatically implemented. For example, recommended parameter settings or automatically implemented parameters may indicate the stimulation to turn on for a certain period of time, and/or to turn off stimulation for a certain period of time. In another example, recommended duty cycle parameter settings may indicate stimulation to turn on for a period of time without creating desensitization of the stimulation. In one or more examples, the recommended parameter settings may indicate stimulation to occur at a certain time of day, for example when the patient is typically awake or active, or sleeping. In one or more examples, recommended parameter settings relate to when the patient has a certain posture, for example when the patient is in a supine position.

In one or more examples, developing the correlation index includes sensing and storing a base line blood flow for a patient without electrical stimulation (602). The base line blood flow is sensed from tissue prior to delivering stimulation. In an example, the baseline blood flow may include stored patient information prior to stimulation when the patient is at rest or active. After the base line blood flow is collected, stimulation is delivered to the patient at a set of stimulation parameters (604), where the stimulation parameters include electrode combination, a polarity of each selected electrode, a voltage or current amplitude, a pulse width, and/or a pulse frequency. Upon delivery of the stimulation at the stimulation parameter, blood flow is sensed for the stimulation parameter and blood flow information is collected (606). Patient information is collected while the patient is being stimulated using the current parameter setting, where the patient information may include activity level, posture, glucose level, body temperature, time of day, diet, input from patient sensors, such as accelerometers (608). In some examples, patient information may include a level of pain perception or perception of stimulation, for example whether the patient feels the stimulation. The blood flow information and patient information are stored with the stimulation parameters within the correlation index (610). The stimulation parameters are modified (610), and the process is repeated to generate additional data to populate the correlation index.

The correlation index may further include a ranking of parameter candidates by program, such as blood flow maximum, pain relief, or energy savings. For example, data in the index is grouped by greatest changes in blood flow, the greatest changes in patient pain rating, or best energy savings. In some examples, sets of parameter settings may be grouped for achieving 25% or greater change in blood flow from the baseline blood flow, 50% change, or 100% change. In some examples, a clinician may set a program to prioritize these groupings for closed loop control. In some examples, the sets of parameter settings may be grouped for patient perception of pain treatment, such as lowest pain rating as correlated to stimulation parameters, medium pain rating, or high pain rating. In some examples, the sets of parameter settings may be grouped by achieving low, medium or high energy conservation. The correlated data may be used by a closed loop control system to implement the most effective changes, while providing the patient the most significant pain relief.

The following numbered examples may illustrate one or more aspects of this disclosure:

Example 1. A system comprising one or more processors configured to: direct delivery of electrical stimulation to a patient, receive information relating to blood flow associated with tissue of the patient upon the delivery of the electrical stimulation to the patient, and generate output based on the received information.

Example 2. The system of example 1, further comprising a user interface, wherein the one or more processors are configured to generate the output based on the received information via the user interface.

Example 3. The system of example 1 or 2, wherein the output comprises one or more blood flow values.

Example 4. The system of any of examples 1-3, wherein the output comprises one or more electrical stimulation efficacy indications for the delivered electrical stimulation.

Example 5. The system of any of examples 1-4, wherein the output comprises one or more recommended electrical stimulation parameters for the delivery of the electrical stimulation.

Example 6. The system of example 5, wherein the one or more recommended parameters include one or more of electrode combination, stimulation amplitude, stimulation pulse width, stimulation frequency, or duty cycle.

Example 7. The system of any of examples 1-6, further comprising:

a blood flow sensing device configured to sense the blood flow associated with the tissue of the patient.

Example 8. The system of example 7, wherein the blood flow sensing device includes at least one of an external blood flow sensor or an implantable blood flow sensor.

Example 9. The system of 7 or 8, further comprising: electrical stimulation circuitry configured to generate the electrical stimulation, and electrodes configured to deliver the electrical stimulation to the patient.

Example 10. The system of example 9, wherein the electrical stimulation circuitry resides in an implantable housing and the blood flow sensing device is housed separately from the electrical stimulation circuitry.

Example 11. The system of example 10, wherein the blood flow sensing device includes an external blood flow sensor.

Example 12. The system of any of examples 7-11, further comprising a telemetry circuitry configured to receive the information from the blood flow sensing device via wireless telemetry.

Example 13. The system of any of examples 1-8, further comprising: electrical stimulation circuitry configured to generate the electrical stimulation, and electrodes configured to deliver the electrical stimulation to the patient.

Example 14. The system of any of examples 1-13, wherein the received information relates to blood flow associated with first tissue of the patient, and the one or more processors are configured to direct delivery of the electrical stimulation to second tissue different than the first tissue.

Example 15. The system of any of examples 1-14, wherein the one or more processors are further configured to: receive user input selecting one or more stimulation parameters of the electrical stimulation, and direct delivery of the electrical stimulation based on the selected parameters.

Example 16. The system of any of examples 1-15, wherein the one or more processors are further configured to: direct delivery of the electrical stimulation based on multiple sets of stimulation parameters, wherein the received information relates to blood flow associated with the tissue of the patient upon the delivery of the electrical stimulation to the patient based on each of the multiple sets of stimulation parameters.

Example 17. The system of example 16, wherein the output comprises respective blood flow values for each of the multiple sets of stimulation parameters.

Example 18. The system of example 16, wherein the output comprises electrical stimulation efficacy indications for the delivered electrical stimulation based on the respective blood flow values for each of the multiple sets of stimulation parameters.

Example 19. The system of example 16, wherein the output comprises one or more recommended electrical stimulation parameters for the delivery of the stimulation based on the respective blood flow values for each of the multiple sets of stimulation parameters.

Example 20. The system of example 19, wherein the one or more recommended stimulation parameters include one or more of electrode combination, stimulation amplitude, stimulation pulse width, stimulation frequency, or duty cycle.

Example 21. The system of any of examples 16-20, wherein the one or more processors are configured to store indications of the received information in association with the multiple sets of stimulation parameters.

Example 22. The system of example 1, further comprising: an implantable medical device comprising: electrical stimulation circuitry configured to generate the electrical stimulation, and electrodes configured to deliver the electrical stimulation to the patient, and a blood flow sensing device configured to sense the blood flow associated with the tissue of the patient and transmit the information to the one or more processors.

Example 23. The system of example 22, wherein the blood flow sensing device is configured to attach to an appendage of the patient to sense blood flow associated with the appendage.

Example 24. The system of any of examples 21-23, further comprising an external programmer including a user interface device and the one or more processors, wherein the one or more processors are configured to generate the output via the user interface.

Example 25. The system of example 1, further comprising: an implantable medical device comprising: electrical stimulation circuitry configured to generate the electrical stimulation, and electrodes configured to deliver the electrical stimulation to the patient, and a control device comprising the one or more processors, the control device configured to: direct delivery of the electrical stimulation to the patient by the implantable medical device with multiple sets of stimulation parameters, receive information relating to blood flow associated with the tissue of the patient upon the delivery of the electrical stimulation to the patient based on each of the multiple sets of parameters, and generate, as at least part of the output, multiple sets of stimulation parameters for the delivery of the electrical stimulation based on the received information.

Example 26. The system of example 25, wherein, to direct delivery of the electrical stimulation to the patient by the implantable medical device with multiple sets of stimulation parameters, the one or more processors are configured to: direct delivery of the electrical stimulation to the patient by the implantable medical device with multiple sets of stimulation parameters, receive first information relating to first blood flow associated with the tissue of the patient upon the delivery of the electrical stimulation to the patient with the first set of stimulation parameters, adjust at least one of the first set of stimulation parameters to create a second set of stimulation parameters, and direct delivery of the electrical stimulation to the patient with the second set of stimulation parameters.

Example 27. The system of example 26, further comprising: a user interface device, wherein the control device is configured to: generate output based on the first received information and the second received information via the user interface device, receive user input via the user interface device, following generation of the output based on the first received information and the second received information, selecting one or more stimulation parameters for the delivery of the electrical stimulation, and generate the third set stimulation parameters for delivery of the electrical stimulation based on the user input.

Example 28. The system of example 26, wherein the control device is configured to: compare the first information relating to the first blood flow with the second information relating to the second blood flow, and automatically generate the third set of stimulation parameters for delivery of the electrical stimulation based on the comparison.

Example 29. The system of any of examples 25-28, wherein the control device is configured to direct delivery of the electrical stimulation to the patient with the third set of stimulation parameters.

Example 30. The system of any of examples 25-28, wherein the one or more stimulation parameters include one or more of electrode combination, stimulation amplitude, stimulation pulse width, stimulation pulse rate, or duty cycle.

Example 31. The system of any of examples 25-28, wherein the control device is configured to automatically direct delivery of the electrical stimulation to the patient by the implantable medical device with the first and second sets of stimulation parameters.

Example 32. The system of any examples 1-31, wherein the electrical stimulation includes one or more stimulation parameters selected to deliver spinal cord stimulation.

Example 33. The system of any of examples 1-31, wherein the electrical stimulation includes one or more stimulation parameters selected to deliver therapy to address a condition of one or more of painful diabetic neuropathy (PDN), peripheral vascular disease (PVD), peripheral artery disease (PAD), complex regional pain syndrome (CRPS), angina pectoris (AP), leg pain, back pain or pelvic pain.

Example 34. The system of any of examples 1-33, wherein the output includes blood flow values.

Example 35. The system of any of examples 1-34, wherein one or more processors are configured to generate additional output including one or more of patient-indicated symptom relief, patient glucose level, patient activity level, patient posture, stimulation energy efficiency.

Example 36. The system of example 35, wherein the one or more processors are configured to present the output on a display device.

Example 37. The system of any of examples 1-36, wherein the one or more processors are configured to generate a correlation index that indexes the received information to one or more stimulation parameters of the electrical stimulation.

Example 38. The system of any of examples 1-36, further comprising a storage device storing data defining a correlation index defining a relationship between blood flow information and parameter information for delivery of the electrical stimulation, wherein the processor circuitry automatically adjusts one or more of the stimulation parameters of the electrical stimulation based on the relationship and automatically controls the electrical stimulation based on the adjusted stimulation parameters.

Example 39. The system of example 38, wherein the parameter information includes one or more stimulation parameters or stimulation parameter adjustments.

Example 40. The system of example 38, wherein the blood flow information includes a differential between sensed blood flow values and target blood flow values, and the parameter information includes stimulation parameter adjustments.

Example 41. A method comprising: directing delivery of electrical stimulation with one or more processors to a patient, receiving information relating to blood flow associated with tissue of the patient upon the delivery of the electrical stimulation to the patient, and generating output based on the received information.

Example 42. The method of example 41, wherein the one or more processors generate the output based on the received information via a user interface.

Example 43. The method of example 41 or 42, wherein the output comprises one or more blood flow values.

Example 44. The method of any of examples 41-43, wherein the output comprises one or more electrical stimulation efficacy indications for the delivered electrical stimulation.

Example 45. The method of any of examples 41-44, wherein the output comprises one or more recommended electrical stimulation parameters for the delivery of the electrical stimulation.

Example 46. The method of example 45, wherein the one or more recommended parameters include one or more of electrode combination, stimulation amplitude, stimulation pulse width, stimulation frequency, or duty cycle.

Example 47. The method of any of examples 41-46, further comprising: sensing blood flow associated with the tissue of the patient with a blood flow sensing device.

Example 48. The method of example 47, wherein sensing blood flow with the blood flow sensing device includes sensing blood flow with at least one of an external blood flow sensor or an implantable blood flow sensor.

Example 49. The method any of examples 41-48, further comprising: generating the electrical stimulation with electrical stimulation circuitry, and delivering the electrical stimulation to the patient with electrodes.

Example 50. The method of example 47, wherein the electrical stimulation circuitry resides in an implantable Example housing and the blood flow sensing device is housed separately from the electrical stimulation circuitry.

Example 51. The method of example 50, wherein the blood flow sensing device includes an external blood flow sensor.

Example 52. The method of any of examples 44-48, wherein receiving information from the blood flow sensing device includes wireless receiving the information via wireless telemetry.

Example 53. The method of any of examples 41-52, wherein the received information relates to blood flow associated with first tissue of the patient, and the one or more processors directing delivery of the electrical stimulation to second tissue different than the first tissue.

Example 54. The method of any of examples 41-53, further comprising: receiving user input selecting one or more stimulation parameters of the electrical stimulation, and directing delivery of the electrical stimulation based on the selected stimulation parameters.

Example 55. The method of any of examples 41-54, further comprising: directing delivery of the electrical stimulation with the processor based on multiple sets of stimulation parameters, wherein the received information relates to blood flow associated with the tissue of the patient upon the delivery of the electrical stimulation to the patient based on each of the multiple sets of stimulation parameters.

Example 56. The method of example 55, wherein the output comprises respective blood flow values for each of the multiple sets of stimulation parameters.

Example 57. The method of example 55, wherein the output comprises electrical stimulation efficacy indications for the delivered electrical stimulation based on the respective blood flow values for each of the multiple sets of stimulation parameters.

Example 58. The method of example 55, wherein the output comprises one or more recommended electrical stimulation parameters for the delivery of the stimulation based on the respective blood flow values for each of the multiple sets of stimulation parameters.

Example 59. The method of example 58, wherein the one or more recommended parameters include one or more of electrode combination, stimulation amplitude, stimulation pulse width, stimulation frequency, or duty cycle.

Example 60. The method of any of examples 55-59, further comprising storing indications of the received information in association with the multiple sets of stimulation parameters.

Example 61. The method of example 41, further comprising: electrical stimulation circuitry of an implantable medical device generating the electrical stimulation, and electrodes of the implantable medical device delivering the electrical stimulation to the patient, and sensing the blood flow associated with the tissue of the patient with a blood flow sensing device and transmitting the information to the one or more processors.

Example 62. The method of example 61, further comprising attaching the blood flow sensing device to an appendage of the patient and sensing blood flow associated with the appendage.

Example 63. The method of any of examples 60-62, further comprising generating the output via an external control device including a user interface device and the one or more processors.

Example 64. The method of example 41, further comprising: generating the electrical stimulation with electrical stimulation circuitry of an implantable medical device, delivering electrical stimulation to the patient with electrodes, directing delivery of the electrical stimulation to the patient by the implantable medical device with multiple sets of stimulation parameters via the control device, receiving information relating to blood flow associated with the tissue of the patient upon the delivery of the electrical stimulation to the patient based on each of the multiple sets of stimulation parameters, and generating with the control device, as at least part of the output, a third set of stimulation parameters for the delivery of the electrical stimulation based on the received information.

Example 65. The method of example 64, further comprising: directing delivery of the electrical stimulation to the patient by the implantable medical device with multiple sets of stimulation parameters, receiving first information relating to first blood flow associated with the tissue of the patient upon the delivery of the electrical stimulation to the patient with the first set of stimulation parameters, adjusting at least one of the first set of stimulation parameters to create a second set of stimulation parameters, and directing delivery of the electrical stimulation to the patient with the second set of stimulation parameters.

Example 66. The method of example 65, further comprising: generating output using the control device based on the first received information and the second received information via a user interface device, receiving user input via the user interface device, following generation of the output based on the first received information and the second received information, selecting one or more stimulation parameters for the delivery of the electrical stimulation, and generating the third set stimulation parameters for delivery of the electrical stimulation based on the user input.

Example 67. The method of example 65, further comprising: comparing the first information relating to the first blood flow with the second information relating to the second blood flow, and automatically generating the third set of stimulation parameters for delivery of the electrical stimulation based on the comparison.

Example 68. The method of any of examples 64-67, further comprising directing delivery of the electrical stimulation to the patient with the third set of stimulation parameters.

Example 69. The method of any of examples 64-67, wherein the one or more stimulation parameters include one or more of electrode combination, stimulation amplitude, stimulation pulse width, stimulation pulse rate, or duty cycle.

Example 70. The method of any of examples 64-67, further comprising automatically directing delivery of the electrical stimulation to the patient by the control device of the implantable medical device with the first and second sets of stimulation parameters.

Example 71. The method of any examples 41-70, wherein the electrical stimulation includes one or more stimulation parameters selection to deliver spinal cord stimulation.

Example 72. The method of any of examples 41-70, wherein the electrical stimulation includes one or more stimulation parameters selection to deliver therapy to address a condition of one or more of painful diabetic neuropathy (PDN), peripheral vascular disease (PVD), peripheral artery disease (PAD), complex regional pain syndrome (CRPS), angina pectoris (AP) leg pain, back pain or pelvic pain.

Example 73. The method of any of examples 41-72, wherein generating the output includes generating blood flow values.

Example 74. The system of any of examples 41-73, further comprising generating additional output including one or more of patient-indicated symptom relief, patient glucose level, patient activity level, patient posture, stimulation energy efficiency.

Example 75. The system of example 74, further comprising presenting the output on a display device.

Example 76. The method of any of examples 41-75, further comprising generating a correlation index with the one or more processors that indexes the received information to one or more stimulation parameters of the electrical stimulation.

Example 77. The method of examples 41-75, further comprising storing data defining a correlation index defining a relationship between blood flow information and parameter information for delivery of the electrical stimulation, wherein the processor circuitry automatically adjusts one or more of the parameters of the electrical stimulation based on the relationship automatically controls the electrical stimulation based on the adjusted parameters.

Example 78. A system comprising: an implantable medical device comprising: electrical stimulation circuitry configured to generate the electrical stimulation, electrodes configured to deliver the electrical stimulation to the patient, a blood flow sensing device configured to sense the blood flow associated with the tissue of the patient, and a controller configured to: direct delivery of the electrical stimulation to the patient by the implantable medical device, receive information from the blood flow sensing device relating to blood flow associated with the tissue of the patient upon the delivery of the electrical stimulation to the patient.

Example 79. The system of example 78, wherein the controller is configured to receive blood flow values from the blood flow sensing device.

Example 80. The system of any of examples 78-79, wherein the controller is configured to send instructions to the blood flow sensing device.

Example 81. The system of any of examples 78-80, wherein the controller is configured to send instructions to the IMD regarding electrical stimulation based on communication from the blood flow sensing device.

Example 82. The system of any of examples 78-81, wherein the electrical stimulation includes one or more stimulation parameters selection to deliver therapy to address a condition of one or more of painful diabetic neuropathy (PDN), peripheral vascular disease (PVD), peripheral artery disease (PAD), complex regional pain syndrome (CRPS), angina pectoris (AP), leg pain, back pain or pelvic pain.

Example 83. A system comprising: electrical stimulation circuitry configured to generate electrical stimulation, electrodes configured to deliver the electrical stimulation to a patient, and processing circuitry configured to: receive information relating to blood flow associated with tissue of the patient, and control the electrical stimulation circuitry to deliver the electrical stimulation to the patient based on the received information.

Example 84. The system of example 83, further comprising: a blood flow sensing device configured to sense the blood flow associated with the tissue of the patient.

Example 85. The system of example 84, wherein the blood flow sensing device includes at least one of an external blood flow sensor or an implantable blood flow sensor.

Example 86. The system of any of examples 84-85, wherein the electrical stimulation circuitry resides in an implantable housing and the blood flow sensing device is housed separately from the electrical stimulation circuitry and the processing circuitry.

Example 87. The system of any of examples 84-86, further comprising a telemetry circuitry configured to receive the information from the blood flow sensing device via wireless telemetry.

Example 88. The system of any of examples 83-87, wherein the received information relates to blood flow associated with a first tissue of the patient, and the processing circuitry is configured to direct delivery of the electrical stimulation to a second tissue different than the first tissue.

Example 89. The system of any of examples 83-88, wherein the processing circuitry is further configured to: adjust one or more stimulation parameters of the electrical stimulation based on the received information, and control the electrical stimulation circuitry to deliver the electrical stimulation based on the adjusted stimulation parameters.

Example 90. The system of any of examples 83-88, wherein the processing circuitry is further configured to: adjust one or more stimulation parameters of the electrical stimulation if the received information indicates a blood flow value is outside a range of blood flow values, is below a minimum blood flow value, or exceeds a maximum blood flow value, and control the electrical stimulation circuitry to deliver the electrical stimulation based on the adjusted stimulation parameters.

Example 91. The system of any of examples 83-90, further comprising a storage device storing data defining a correlation index defining a relationship between blood flow information and parameter information for delivery of the electrical stimulation, wherein the processor circuitry automatically adjusts one or more of the stimulation parameters of the electrical stimulation based on the relationship defined by the correlation index.

Example 92. The system of example 91, wherein the blood flow information is the received information.

Example 93. The system of any of examples 91-92, wherein the parameter information includes one or more electrical stimulation parameters or parameter adjustments.

Example 94. The system of example 91, wherein the blood flow information includes a difference between sensed blood flow values and target blood flow values, and the parameter information includes electrical stimulation parameter adjustments.

Example 95. The system of examples 89-94, wherein the one or more stimulation parameters include one or more of electrode combination, stimulation amplitude, stimulation pulse width, stimulation pulse rate, or duty cycle.

Example 96. The system of any one of examples 83-95, wherein the processing circuitry is configured further to receive multiple instances of the information relating to blood flow associated with the tissue of the patient substantially continuously over time.

Example 97. The system of any one of examples 83-95, wherein the processing circuitry is configured further to receive multiple instances of the information relating to blood flow associated with the tissue of the patient intermittently over time.

Example 98. The system of any one of examples 83-97, wherein the processing circuitry is configured further to receive multiple instances of the information relating to blood flow associated with the tissue of the patient responsive to events occurring over time.

Example 99. The system of any of examples 83-98, wherein the processing circuitry is configured to control the electrical stimulation circuitry to deliver the electrical stimulation to the patient based on multiple instances of received information over time.

Example 100. The system of example 83, further comprising: an implantable stimulator including the electrical stimulation circuitry configured to generate the electrical stimulation electrodes configured to deliver the electrical stimulation to the patient, and a blood flow sensing device configured to sense the blood flow associated with the tissue of the patient and transmit the information to the processing circuitry.

Example 101. The system of example 100, wherein the electrical stimulation circuitry is configured to generate the electrical stimulation with one or more parameters selected for delivery by the electrodes as spinal cord stimulation.

Example 102. The system of examples 100 or 101, wherein the electrical stimulation includes one or more parameters selected to deliver therapy to address a condition of one or more of painful diabetic neuropathy (PDN), peripheral vascular disease (PVD), peripheral artery disease (PAD), complex regional pain syndrome (CRPS), angina pectoris (AP), leg pain, back pain or pelvic pain.

Example 103. The system of any of examples 100-102, wherein the blood flow sensing device is configured to attach to an appendage of the patient to sense blood flow associated with the appendage.

Example 104. The system of any of examples 83-103, further comprising: an implantable medical device including the electrodes configured to deliver the electrical stimulation to the patient, and

an external controller wherein at least a portion of the processing circuitry is within the external controller.

Example 105. The system of example 104, wherein at least a portion of the processing circuitry is in the implantable medical device.

Example 106. The system of any of examples 83-103, further comprising: an implantable medical device including the electrical stimulation circuitry and the electrodes, wherein the processing circuitry is in the implantable medical device.

Example 107. A method comprising: generating electrical stimulation with electrical stimulation circuitry, delivering the electrical stimulation with electrodes to a patient, receiving information relating to blood flow associated with tissue of the patient upon delivering the electrical stimulation to the patient, and controlling the electrical stimulation circuitry to deliver the electrical stimulation to the patient based on the received information.

Example 108. The method of example 107, further comprising sensing blood flow associated with the tissue of the patient with a blood flow sensing device.

Example 109. The method of example 108, wherein the blood flow sensing device includes at least one of an external blood flow sensor or an implantable blood flow sensor.

Example 110. The method of any of examples 108-109, wherein the electrical stimulation circuitry resides in an implantable housing and the blood flow sensing device is housed separately from the electrical stimulation circuitry and the processing circuitry.

Example 111. The method of any of examples 108-110, further comprising receiving the information from the blood flow sensing device via wireless telemetry.

Example 112. The method of any of examples 107-111, wherein the received information relates to blood flow associated with a first tissue of the patient, and the processing circuitry is configured to direct delivery of the electrical stimulation to second tissue different than the first tissue.

Example 113. The method of any of examples 107-112, further comprising: adjusting one or more stimulation parameters of the electrical stimulation based on the received information, and controlling the electrical stimulation circuitry to deliver the electrical stimulation based on the adjusted stimulation parameters.

Example 114. The method of any of examples 107-112, further comprising: adjusting one or more stimulation parameters of the electrical stimulation if the received information indicates a blood flow value is outside of a range of blood flow values, is below a minimum blood flow value, or exceeds a maximum blood flow value, and controlling the electrical stimulation circuitry to deliver the electrical stimulation based on the adjusted stimulation parameters.

Example 115. The method of any of examples 107-114, further comprising storing data on a storage device, the data defining a correlation index defining a relationship between blood flow information and parameter information for delivery of the electrical stimulation, wherein the processor circuitry automatically adjusts one or more of the stimulation parameters of the electrical stimulation based on the relationship defined by the correlation index.

Example 116. The method of example 115, wherein the blood flow information is the received information.

Example 117. The method of any of examples 115-116, wherein the parameter information includes one or more electrical stimulation parameters or stimulation parameter adjustments.

Example 118. The method of example 115, wherein the blood flow information includes a differential between sensed blood flow values and target blood flow values, and the parameter information includes electrical stimulation parameter adjustments.

Example 119. The method of examples 113-118, wherein the one or more stimulation parameters include one or more of electrode combination, stimulation amplitude, stimulation pulse width, stimulation pulse rate, or duty cycle.

Example 120. The method of any one of examples 107-119, further comprising receiving multiple instances of the information relating to blood flow associated with the tissue of the patient substantially continuously over time.

Example 121. The method of any one of examples 107-119, further comprising receiving multiple instances of the information relating to blood flow associated with the tissue of the patient intermittently over time.

Example 122. The method of any one of examples 107-121, wherein the processing circuitry is configured further to receive multiple instances of the information relating to blood flow associated with the tissue of the patient responsive to events occurring over time.

Example 123. The method of any of examples 107-122, further comprising controlling the electrical stimulation circuitry to deliver the electrical stimulation to the patient based on multiple instances of received information over time.

Example 124. The method of example 107, further comprising: generating the electrical stimulation with an implantable stimulator, delivering electrical stimulation with electrodes, and sensing the blood flow associated with the tissue of the patient with a blood flow sensing device.

Example 125. The method of example 124, wherein generating the electrical stimulation with one or more stimulation parameters selected for delivery by the electrodes is spinal cord stimulation.

Example 126. The method of examples 124 or 125, wherein the electrical stimulation includes one or more stimulation parameters selected to deliver therapy to address a condition of one or more of painful diabetic neuropathy (PDN), peripheral vascular disease (PVD), peripheral artery disease (PAD), complex regional pain syndrome (CRPS), angina pectoris (AP), leg pain, back pain or pelvic pain.

Example 127. The method of any of examples 124-126, further comprising attaching the blood flow sensing device to an appendage of the patient to sense blood flow associated with the appendage.

Example 128. The method of any of examples 107-127, further comprising: an implantable medical device including the electrodes configured to deliver the electrical stimulation to the patient, and

an external controller wherein at least a portion of the processing circuitry is within the external controller.

Example 129. The method of example 128, wherein at least a portion of the processing circuitry is in the implantable medical device.

Example 130. The method of any of examples 107-127, further comprising: an implantable medical device including the electrical stimulation circuitry and the electrodes, wherein the processing circuitry is in the implantable medical device.

Example 131. A computer-readable medium comprising instructions to cause one or more processors to perform the method of any of examples 107-130.

The techniques described in this disclosure may be implemented, at least in part, in hardware, software, firmware or any combination thereof. For example, various aspects of the described techniques may be implemented within processing circuitry, which may include one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also form one or more processors or processing circuitry configured to perform one or more of the techniques of this disclosure.

Such hardware, software, and firmware may be implemented, and various operation may be performed within same device, within separate devices, and/or on a coordinated basis within, among or across several devices, to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components. Processing circuitry described in this disclosure, including a processor or multiple processors, may be implemented, in various examples, as fixed-function circuits, programmable circuits, or a combination thereof. Fixed-function circuits refer to circuits that provide particular functionality with preset operations. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive stimulation parameters or output stimulation parameters), but the types of operations that the fixed-function circuits perform are generally immutable. In some examples, one or more of the units may be distinct circuit blocks (fixed-function or programmable), and in some examples, one or more of the units may be integrated circuits.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

What is claimed is:

1. A system comprising one or more processors configured to:
direct delivery of electrical stimulation to a patient;
receive information relating to blood flow associated with tissue of the patient upon the delivery of the electrical stimulation to the patient; and
generate output based on the received information.

2. The system of claim 1, further comprising a user interface, wherein the one or more processors are configured to generate the output based on the received information via the user interface.

3. The system of claim 1, wherein the output comprises one or more of:
one or more blood flow values;
one or more electrical stimulation efficacy indications for the delivered electrical stimulation; and
one or more recommended electrical stimulation parameters for the delivery of subsequent electrical stimulation.

4. The system of claim 3, wherein the one or more recommended parameters include one or more of electrode combination, stimulation amplitude, stimulation pulse width, stimulation frequency, or duty cycle.

5. The system of claim 1, further comprising:
a blood flow sensing device configured to sense the blood flow associated with the tissue of the patient.

6. The system of claim 5, wherein the blood flow sensing device is configured to attach to an appendage of the patient to sense blood flow associated with the appendage.

7. The system of claim 6, further comprising:
electrical stimulation circuitry configured to generate the electrical stimulation; and
electrodes configured to deliver the electrical stimulation to the patient.

8. The system of claim 7, wherein the electrical stimulation circuitry resides in an implantable housing and the blood flow sensing device is housed separately from the electrical stimulation circuitry.

9. The system of claim 1, wherein the received information relates to blood flow associated with first tissue of the patient, and the one or more processors are configured to direct delivery of the electrical stimulation to second tissue different than the first tissue.

10. The system of claim 1, wherein the one or more processors are further configured to:
direct delivery of the electrical stimulation based on multiple sets of stimulation parameters,
wherein the received information relates to blood flow associated with the tissue of the patient upon the delivery of the electrical stimulation to the patient based on each of the multiple sets of stimulation parameters.

11. The system of claim 10, wherein the output comprises one or more of:
respective blood flow values for each of the multiple sets of stimulation parameters;
electrical stimulation efficacy indications for the delivered electrical stimulation based on the respective blood flow values for each of the multiple sets of stimulation parameters; and
one or more recommended electrical stimulation parameters for the delivery of the stimulation based on the respective blood flow values for each of the multiple sets of stimulation parameters.

12. The system of claim 1, further comprising:
an implantable medical device comprising:
electrical stimulation circuitry configured to generate the electrical stimulation, and
electrodes configured to deliver the electrical stimulation to the patient; and a control device comprising the one or more processors, the control device configured to:
direct delivery of the electrical stimulation to the patient by the implantable medical device with multiple sets of stimulation parameters;
receive information relating to blood flow associated with the tissue of the patient upon the delivery of the electrical stimulation to the patient based on each of the multiple sets of parameters; and
generate, as at least part of the output, multiple sets of stimulation parameters for the delivery of the electrical stimulation based on the received information.

13. The system of claim 12, wherein, to direct delivery of the electrical stimulation to the patient by the implantable medical device with multiple sets of stimulation parameters, the one or more processors are configured to:
receive first information relating to first blood flow associated with the tissue of the patient upon the delivery of the electrical stimulation to the patient with the first set of stimulation parameters;
adjust at least one of the first set of stimulation parameters to create a second set of stimulation parameters; and
direct delivery of the electrical stimulation to the patient with the second set of stimulation parameters.

14. The system of claim 13, wherein the one or more processors are further configured to:
receive second information relating to second blood flow associated with the tissue of the patient upon the delivery of the electrical stimulation to the patient with the second set of stimulation parameters, wherein the system further comprises:
a user interface device,
wherein the control device is configured to:
generate output based on the first received information and the second received information via the user interface device;
receive user input via the user interface device, following generation of the output based on the first received information and the second received information, selecting one or more stimulation parameters for the delivery of the electrical stimulation; and
generate a third set stimulation parameters for delivery of the electrical stimulation based on the user input.

15. The system of claim 13, wherein the control device is configured to:
compare the first information relating to the first blood flow with the second information relating to the second blood flow; and
automatically generate a third set of stimulation parameters for delivery of the electrical stimulation based on the comparison.

16. The system of claim 15, wherein the control device is configured to direct delivery of the electrical stimulation to the patient with the third set of stimulation parameters.

17. The system of claim 12, wherein the control device is configured to automatically direct delivery of the electrical stimulation to the patient by the implantable medical device with the multiple sets of stimulation parameters.

18. The system of claim 1, wherein the electrical stimulation includes one or more stimulation parameters selected to deliver spinal cord stimulation.

19. The system of claim 1, wherein the electrical stimulation includes one or more stimulation parameters selected to deliver therapy to address a condition of one or more of painful diabetic neuropathy (PDN), peripheral vascular disease (PVD), peripheral artery disease (PAD), complex regional pain syndrome (CRPS), angina pectoris (AP), leg pain, back pain or pelvic pain.

20. The system of claim 1, wherein one or more processors are configured to generate additional output including one or more of patient-indicated symptom relief, patient glucose level, patient activity level, patient posture, stimulation energy efficiency.

21. The system of claim 1, wherein the one or more processors are configured to generate a correlation index that indexes the received information to one or more stimulation parameters of the electrical stimulation.

22. The system of claim 1, further comprising a storage device storing data defining a relationship between blood flow information and parameter information for delivery of the electrical stimulation, wherein the processor circuitry automatically adjusts one or more of the stimulation parameters of the electrical stimulation based on the relationship and automatically controls the electrical stimulation based on the adjusted stimulation parameters.

23. The system of claim 22, wherein the parameter information includes one or more stimulation parameters or stimulation parameter adjustments.

24. The system of claim 22, wherein the blood flow information includes a differential between sensed blood flow values and target blood flow values, and the parameter information includes stimulation parameter adjustments.

25. A method comprising:
directing delivery of electrical stimulation to a patient;
receiving information relating to blood flow associated with tissue of the patient upon the delivery of the electrical stimulation to the patient; and
generating output based on the received information.

26. The method of claim 25, wherein generating the output comprises:
generating, via a user interface, the output based on the received information.

27. The method of claim 25, wherein the output comprises one or more of:
one or more blood flow values;
one or more electrical stimulation efficacy indications for the delivered electrical stimulation; and
one or more recommended electrical stimulation parameters for the delivery of subsequent electrical stimulation.

28. The method of claim 27, wherein the one or more recommended parameters include one or more of electrode combination, stimulation amplitude, stimulation pulse width, stimulation frequency, or duty cycle.

29. The method of claim 25, wherein receiving the information relating to the blood flow comprises receiving the information related to the blood flow via a blood flow sensing device configured to sense the blood flow associated with the tissue of the patient.

30. The method of claim 29, wherein the blood flow sensing device is configured to attach to an appendage of the patient to sense blood flow associated with the appendage.

31. The method of claim 30, wherein directing the delivery of the electrical stimulation comprises:
causing electrical stimulation circuitry to generate the electrical stimulation for delivery to the patient via electrodes.

32. The method of claim 31, wherein the electrical stimulation circuitry resides in an implantable housing and the blood flow sensing device is housed separately from the electrical stimulation circuitry.

33. The method of claim 25, wherein the received information relates to blood flow associated with first tissue of the patient, and wherein directing the delivery of the electrical stimulation comprises directing delivery of the electrical stimulation to second tissue different than the first tissue.

34. The method of claim 25, further comprising:
directing delivery of the electrical stimulation based on multiple sets of stimulation parameters,
wherein the received information relates to blood flow associated with the tissue of the patient upon the delivery of the electrical stimulation to the patient based on each of the multiple sets of stimulation parameters.

35. The method of claim 34, wherein the output comprises one or more of:
respective blood flow values for each of the multiple sets of stimulation parameters;
electrical stimulation efficacy indications for the delivered electrical stimulation based on the respective blood flow values for each of the multiple sets of stimulation parameters; and
one or more recommended electrical stimulation parameters for the delivery of the stimulation based on the respective blood flow values for each of the multiple sets of stimulation parameters.

36. The method of claim 25, further comprising:
generating the electrical stimulation with electrical stimulation circuitry of an implantable medical device;
delivering electrical stimulation to the patient with electrodes;
directing delivery of the electrical stimulation to the patient by the implantable medical device with multiple sets of stimulation parameters via the control device;
receiving information relating to blood flow associated with the tissue of the patient upon the delivery of the electrical stimulation to the patient based on each of the multiple sets of stimulation parameters; and
generating with the control device, as at least part of the output, a third set of stimulation parameters for the delivery of the electrical stimulation based on the received information.

37. The method of claim 36, further comprising:
receiving first information relating to first blood flow associated with the tissue of the patient upon the delivery of the electrical stimulation to the patient with the first set of stimulation parameters;
adjusting at least one of the first set of stimulation parameters to create a second set of stimulation parameters; and
directing delivery of the electrical stimulation to the patient with the second set of stimulation parameters.

38. The method of claim 37, further comprising:
generating output using the control device based on the first received information and the second received information via a user interface device;
receiving user input via the user interface device, following generation of the output based on the first received information and the second received information, selecting one or more stimulation parameters for the delivery of the electrical stimulation; and
generating the third set stimulation parameters for delivery of the electrical stimulation based on the user input.

39. The method of claim 37, further comprising:
comparing the first information relating to the first blood flow with the second information relating to the second blood flow; and
automatically generating the third set of stimulation parameters for delivery of the electrical stimulation based on the comparison.

40. The method of claim 36, further comprising directing delivery of the electrical stimulation to the patient with the third set of stimulation parameters.

41. The method of claim 36, further comprising automatically directing delivery of the electrical stimulation to the patient by the control device of the implantable medical device with the first and second sets of stimulation parameters.

42. The method of claim 25, wherein the electrical stimulation includes one or more stimulation parameters selected to deliver spinal cord stimulation.

43. The method of claim 25, wherein the electrical stimulation includes one or more stimulation parameters selected to deliver therapy to address a condition of one or more of painful diabetic neuropathy (PDN), peripheral vascular disease (PVD), peripheral artery disease (PAD), complex regional pain syndrome (CRPS), angina pectoris (AP), leg pain, back pain or pelvic pain.

44. The method of claim 25, further comprising:
generating additional output including one or more of patient-indicated symptom relief, patient glucose level, patient activity level, patient posture, stimulation energy efficiency.

45. The method of claim 25, further comprising:
generating a correlation index that indexes the received information to one or more stimulation parameters of the electrical stimulation.

46. The method of claim 25, further comprising:
automatically adjusting one or more of the stimulation parameters of the electrical stimulation based on a relationship between the blood flow information and parameter information for delivery of the electrical stimulation; and
automatically controlling the electrical stimulation based on the adjusted stimulation parameters.

47. The method of claim 46, wherein the parameter information includes one or more stimulation parameters or stimulation parameter adjustments.

48. The method of claim 46, wherein the blood flow information includes a differential between sensed blood flow values and target blood flow values, and the parameter information includes stimulation parameter adjustments.

49. The method of claim 25, wherein generating the output comprises:
delivering, based on the received information, electrical stimulation to the patient in a closed-loop configuration.

50. The method of claim 24, wherein delivering the electrical stimulation in the closed-loop configuration comprises:
determining, based on the received information, parameters for the subsequent electrical stimulation.
